(19) United States
(12) Patent Application Publication
(10) Pub. No.: US 2024/0173375 A1
(43) Pub. Date: May 30, 2024

(54) CHEMOTACTIC AUTOPHAGY-INHIBITING PEPTIDE, COMPOSITIONS AND METHODS RELATED THERETO

(71) Applicant: Biotempt B.V., The Hague (NL)

(72) Inventor: Gert Wensvoort, Koekange (NL)

(21) Appl. No.: 18/548,845

(22) PCT Filed: Mar. 2, 2022
(86) PCT No.: PCT/NL2022/050115

§ 371 Date: Sep. 1, 2023

(30) Foreign Application Priority Data
Mar. 2, 2021 (EP) 21160152.1

(51) Int. Cl. A61K 38/05; A61K 38/06; A61K 38/07; A61K 38/28; A61K 47/02; A61K 47/10; A61K 47/68; C07K 7/08; C07K 14/00

(52) U.S. Cl. CPC A61K 38/05; A61K 38/06; A61K 38/07; A61K 38/28; A61K 47/02; A61K 47/10; A61K 47/6849; C07K 7/08; C07K 14/001

(57) ABSTRACT

Methods for the treatment of diseases involving autophagy by leukocytes, preferably neutrophil cells, which process, according to the disclosure, is involved in mechanisms of tissue repair, vascular permeability and immune responses. The disclosure provides methods and means to target a chemoattractant receptor, preferably a leukocyte cell-surface receptor specifically and to provide molecules and compositions comprising a specific targeting agent as well as amino acid compositions that are involved in the pathway of autophagy and the diseases related thereto. The disclosure also relates to peptide drug development, in particular, to (the improvement of) autophagy inhibiting amino acid containing peptides, more in particular, glutamine-containing peptides and/or glutamine and other autophagy-modulating amino acid containing compositions useful in the treatment of vascular and inflammatory conditions.

### CROSS-REFERENCE TO RELATED APPLICATIONS

This application is a national phase entry under 35 U.S.C. § 371 of International Patent Application PCT/NL2022/050115, filed Mar. 2, 2022, designating the United States of America and published as International Patent Publication WO 2022/186690 A1 on Sep. 9, 2022, which claims the benefit under Article 8 of the Patent Cooperation Treaty to European Patent Application Serial No. EP 21160152.1, filed Mar. 2, 2021.

### STATEMENT ACCORDING TO 37 C.F.R. § 1.821(c) or (e)-SEQUENCE LISTING SUBMITTED AS A TXT AND PDF FILES

Pursuant to 37 C.F.R. § 1.821(c) or (e), files containing a TXT version and a PDF version of the Sequence Listing have been submitted concomitant with this application, the contents of which are hereby incorporated by reference.

### TECHNICAL FIELD

The disclosure relates to means and methods for the treatment of diseases involving autophagy by leukocytes, preferably neutrophil cells, which process according to the disclosure is involved in mechanisms of tissue repair, vascular permeability and immune responses.

### BACKGROUND

### Chemoattractant Receptors

Since their identification and molecular cloning, a large body of knowledge has accumulated concerning the biological roles, the intracellular signaling, and the regulation of chemoattractant receptors, such as formyl-peptide receptors (FPR), complement receptors and chemokine receptors. Their pathophysiological role has been shown to extend beyond host resistance against microbial infection. The ability of FPR to interact with high affinity with agonists derived from pathogens suggests that this receptor plays a critical role in innate immunity. It has been suggested to behave as a pattern recognition receptor. It is puzzling, but perhaps of pathophysiological relevance, as to how this receptor escapes the classical mode of regulation that applies to its homologue FPRL1, and to C5aR. FPR, and especially FPRL 1, can now be considered as promiscuous receptors, with affinity for apparently unrelated agonists. The use of these chemoattractant receptors by host-derived agonists indicates that this receptor may play a crucial role in the regulation of the inflammatory process associated with tissue damage and degeneration. Therefore, it seems of importance to consider chemoattractant receptors as potential targets in the search for specific anti-inflammatory drugs and for the development of new therapeutic strategies tissue repair, vascular permeability and immune responses. Chemoattractant receptors, including C5aR and the members of the FPR family, are generally coupled to the heterotrimeric G proteins of the Gi subtype as evidenced by the observation that chemoattractant-mediated neutrophil functions, i.e., chemotaxis, degranulation, and superoxide production, are largely inhibited by treatment of cells with pertussis toxin (PTX). Cell responses to chemotactic factors are tightly controlled by up-regulation through priming or down-regulation by desensitization/internalization. C5aR and the N-formyl peptide receptors are structurally and functionally closely related to chemokine receptors. Both homo- and hetero-dimerization were demonstrated for CC and CXC chemokine receptors. A recent study also showed that, for example, the chemokine receptor CCR5 forms hetero-oligomeric complexes with C5aR. As more specific ligands are discovered and the immunological tools refined, FPR family members and C5aR are found to be expressed differently by a variety of cell types and not restricted to phagocytes as previously thought. FPRL2 is mostly present in monocytes/macrophages but not always in neutrophils, whereas FPR, FPRL1, and C5aR are expressed in neutrophils and monocytes/macrophages. Human dendritic cells express FPRL2 and C5aR throughout maturation, whereas FPR is only present in immature dendritic cells. No functional FPRL1 could be detected in either immature or mature dendritic cells. The expression of chemoattractant receptors such as the formyl peptide receptors in a variety of cells other than phagocytic cells suggests that they might have functional roles beyond that of host defense in innate immune response.

Leukocytes and, in particular, neutrophils have a widely varied chemotactic and phagocytic repertoire that helps them extravasate and home in on tissue damage and clean up that damage through phagocytosis. Neutrophils (neutrophilic granulocytes or polymorph nuclear neutrophils (PMNs; immunology.org/public-information/bitesized-immunology/cells/neutrophils) are the most abundant white blood cell in humans. They have distinct roles in tissue regeneration and repair (Cell Tissue Res. 2018; 371(3): 531-539). Neutrophils comprise a large proportion of the early cellular infiltrate in inflamed tissues and are the major constituent of pus. Neutrophils represent the first line of defense in response to invading microbes, by phagocytosis of pathogens and/or release of antimicrobial factors contained in specialized granules. Neutrophils are typically the first white blood cells recruited to sites of acute inflammation, in response to chemoattractant motifs (or chemotactic cues, also termed chemoattractant) such as CXCL8 (interleukin-8, IL-8), complement, antibody, PGP-like peptide motifs, formylated mitochondrial peptides and many other chemoattractant motifs. Such chemoattractant motifs, despite their great variety, serve only one goal, recognition by and attraction of cells, that then generate appropriate responses. The chemo-attractant motifs are herein jointly identified as chemo-attractant motif W.

Phagocytosis is an active, receptor-mediated process during which a proteinaceous substance, for example, a pathogen is internalized into a specialized vacuole, the phagosome. The interaction with the substance or pathogen can be direct, through chemo-attraction via recognition of chemo-attractant motifs such as damage- or pathogen-associated molecular pattern (DAMP/PAMP) receptors, or indirect, through recognition of opsonized microbes/antigens by Fc receptors or complement receptors. Both direct as well as indirect attraction by definition herein is achieved by receptors capable of recognizing a chemo-attractant motif W.

Phagocytosis, uptake of (proteinaceous) substances by immune cells is an important mechanism of the host-defense system and a primary function of immune cells (leukocytes) such as macrophages and neutrophils. It is among others facilitated by opsonization, a process among others seen in the complement-cascade by which protein or peptide components tag pathogens or tissue derived proteinaceous substances for recognition by leukocytes such as neutrophils and macrophages, mediating chemo-attraction trough binding of such substances to cell-surface receptors of the complement receptor family, after which such substances are taken up by the immune cells. An opsonin is any molecule that enhances chemo-attraction and subsequent phagocytosis by marking an antigen for an immune response or marking dead cells for recycling. Opson in ancient Greece referred to the delicious side-dish of any meal, versus the sitos, or the staple of the meal. Two major roles of complement are to control certain bacterial infections and to promote clearance of apoptotic cells and other substances from injured tissues. Kawatsu et al. (Journal of Pharmacology and Experimental Therapeutics July 1996, 278 (1) 432-440) describe a panel of conformationally constrained, decapeptide agonists corresponding to the C-terminal “effector” region of human C5a (C5a65-74 or ISHKDMQLGR (SEQ IDNO:1)) that was evaluated for the ability to increase vascular permeability. One constrained analog, acyl-YSFKPMPLaR (SEQ ID NO:14), expressed between 2 and 10% of full C5a activity in increasing vascular permeability, as measured by the extravasation of Evans blue dye in guinea pig skin. This analog was at least 10-fold more potent than its unconstrained sister analog C5a65-74465, F67++(YSFKDMQLGR (SEQ ID NO:3)), which was used as an internal standard in these assays.

Similarly, FC-receptors on the surface of leukocytes have an ability of specific binding for a part of an antibody known as the Fc fragment region. Fc receptors are found on the membrane of certain immune cells, including B lymphocytes, natural killer cells, macrophages, neutrophils, and mast cells. Fc receptors binding to antibodies that are attached to infected cells or invading pathogens leads to the protective functions of the immune system. Their activity stimulates phagocytic or cytotoxic cells to destroy microbes, or infected cells by antibody-mediated phagocytosis or other antibody-dependent cell-mediated cytotoxicity.

Other cell-surface receptors involved in binding and uptake of proteinaceous substances are found among the so-called “seven transmembrane” (7TM) receptors, a large family of proteins with a common motif of seven groups of 20-24 hydrophobic amino acids arranged as α-helices (doi.org/10.1111/j.1476-5381.2011.01649_3.x). Approximately 800 of these seven transmembrane (7TM) receptors have been identified of which over 300 are non-olfactory receptors).

The nomenclature of 7TM receptors is commonly used interchangeably with G protein-coupled receptors (GPCR), although the former nomenclature recognises signalling of 7TM receptors through pathways not involving G proteins. The 300+ non-olfactory GPCR are the targets for the majority of drugs in clinical usage although only a minority of these receptors are exploited therapeutically.

Seven transmembrane receptors (7TMRs; Pharmacol. Rev. 2010 Jun.; 62(2): 265-304) are molecules, situated as intrinsic plasma membrane proteins, that bind to natural ligands approaching from one milieu (extracellular) and respond by activating signaling cascades emanating from molecular interactions in a distinct (cytosolic) milieu. Their fundamental nature requires extracellular ligand binding to result in a dynamic change in receptor conformation that is reflected in exposure of a signaling domain at the cytosolic surface, which interacts with the classic proximal effecter partner, a heterotrimeric G protein. However, not only are these regions of classic function important, but they also provide their respective regions for the binding of allosteric ligands from the extracellular space and the cytosol. In addition, the intramembranous surfaces of 7TMRs within the plane of the membrane provide still more sites for possible allosteric action. These three allosteric vectors, directed toward 1) the ectodomain, 2) the cytosolic face, and 3) the intramembranous faces of 7TMRs, provide numerous opportunities for functional selectivity of the action of drugs (see section V.C.2.c). Chemokine signaling is known to be particularly pleiotropic with chemokines showing cross-reactivity to a number of chemokine receptor types, leading to a redundancy of receptor activities and a robust output (Immunol. Today. 1999 Jun.; 20(6):254-7; Trends Pharmacol. Sci. 2006 Jan.; 27(1):41-7.). Also, in cases in which more than one chemokine receptor is targeted in inflammatory disease, the production of receptor dimers can confer sensitivity of multiple receptor types for a single antagonist. Such effects have among others been noted for CCR2/CXCR4 receptor heterodimers (J. Biol. Chem. 2007 Oct. 12; 282(41):30062-9.).

The chemokine receptor CXCR4 (J. Biol. Chem. 2003 Jan. 10; 278(2):896-907.) is a co-receptor for T-tropic strains of human immunodeficiency virus (HIV). Two peptides, designated RSVM (SEQ ID NO:4) and ASLW (SEQ ID NO:5), were identified as novel agonists that are insensitive to the CXCR4 antagonist AMD3100. In chemotaxis assays using the acute lymphoblastic leukemia cell line CCRF-CEM, RSVM (SEQ ID NO:4) behaves as a partial agonist and ASLW (SEQ ID NO:5) as a superagonist. These results suggest that alternative agonist-binding sites are present on CXCR4 that could be screened to develop molecules for therapeutic use, for example, in the treatment of HIV-infections. Typical neutrophil chemokine receptors that mediate chemotaxis and that allow modulation of bioactivity of neutrophils are fMLP-, C5a and/or ELR-positive CXC chemokine-receptors (Infect. Immun. 2000 Oct.; 68(10): 5908-5913, frontiersin.org/articles/10.3389/fimmu.2017.00464/full) located on the surface of neutrophils, through which chemotaxis of neutrophils may be induced.

Neutrophils are the first white blood cells recruited to sites of acute inflammation, in response to chemotactic cues (also termed chemoattractant) such as CXCL8 (interleukin-8, IL-8), complement, antibody or formylated mitochondrial peptides such as fMLP produced by mitochondria in stressed tissue cells and tissue-resident immune cells such as macrophages, and by bacteria. Typical peptide ligand chemoattractant motifs through which binding is achieved also comprise so-called PGP-peptides that arise from exposed stressed and damaged extracellular matrix collagens.

After trauma, cellular injury releases endogenous damage-associated molecular patterns (DAMPs) that activate the innate immune system. Mitochondrial DAMPs express at least two molecular signatures, N-formyl peptides and mitochondrial DNA that act on formyl peptide receptors (FPRs) and Toll-like receptor 9, respectively. Formyl-peptide receptors (FPRs) are a family of seven transmembrane domains, Gi-protein-coupled receptors (GPCRs). In human, there are 3 FPRs, FPR1, FPR2 and FPR3. FPR1 and FPR2 were originally identified based on their capacity to recognize N-formyl peptides produced in nature by degradation of either bacterial or host cell mitochondrial proteins, which represent major proinflammatory products. Activation of FPR1 and FPR2 by chemotactic agonists elicits a cascade of signaling events leading to myeloid cell migration, mediator release, increased phagocytosis and new gene transcription. But for FPR3, although it is expressed in monocytes and dendritic cells (DCs), the overall function remains unclear. The formylpeptide receptors (ENSFM00510000502765, nomenclature agreed by NC-IUPHAR Subcommittee on the formyl peptide receptor family) respond to exogenous ligands such as the bacterial product N-formyl-Met-Leu-Phe (fMLF) and endogenous ligands such as annexin I, cathepsin G and spinorphin, derived from β-hemoglobin. Though some of the diverse FPR ligands are small-molecules or non-peptides, the majority are small peptides that are either synthetic or natural with origins ranging from host and multicellular organisms to viruses and bacteria. These peptides have been extensively studied and patterns of recognized elements have begun to emerge. The presence of formylated methionine in the peptide is generally an activator of FPR1, while FPR2 is less dependent upon this particular residue. Expanding upon this it was concluded that FPR recognition of bacterial peptides requires either a formylated methionine at the N-terminus or an amidated methionine at the C-terminus of a peptide, though it is believed that as a general principle, the secondary structure rather than the primary sequence is important for recognition of the highly diverse ligands by FPR.

While formylated peptides first drew the attention of the scientific community to FPRs, there are many other bacterial/viral peptides that are not necessarily formylated but that nevertheless elicit receptor responses (Int. J. Mol. Sci. 2019, 20(14), 3426). Although the majority of formylated microbial peptides preferentially activate FPR1, the preferred receptor for non-formylated peptides is FPR2. A large percentage of these non-formylated microbe-derived peptides are viral, and many of them are derived from the Human Immunodeficiency Virus (HIV) envelope proteins, including gp41 T20/DP178, gp41 T21/DP107, gp120 V3 loop, gp41 N36, gp120 F, and gp41 MAT-1. Despite the potential importance of FPRs in HIV research, very little work has been done to further explore this connection. It has also been demonstrated that persistent FPR activation desensitized host CCR5 and CXCR4 co-receptors to HIV proteins, thus reducing viral entry and subsequent replication. Still other viruses, including Hepatitis C Virus, coronavirus, and Herpes Simplex Virus, produce chemotactic ligands C5a, N-formyl coronavirus peptide, and gG-2p20, respectively, for FPR1 or FPR2 activation. There is, however, some argument as to the efficacy of the Herpes Simplex viral peptide as an FPR agonist, as the overlapping sequence gG-2p19 was unable to definitively demonstrate that FPR activation played a significant role in the NK response to this virus. Mills (Biochim. Biophys. Acta 2006, 1762, 693-703) used the sequence homology of T20/DP178 to further determine that the OC43 Coronavirus, 229E Coronavirus, NL36 Coronavirus, and even the Ebola Spike Protein were all peptides with aromatic-rich domains that elicited FPR-dependent cell activation. Interestingly, when examined from the context of the FPRs rather than the ligands, it was found that domain variability in the receptors determined ligand binding and subsequent cellular responses. This led to the conclusion that the variability of receptors among individuals might predispose or protect against certain viral infections, the susceptibility of which may be determined by receptor activation. Certain peptides from different strains of Enterococcus faecium have demonstrated FPR activation properties, though the ligand activity is not entirely predictable based on structure. Interestingly, E. faecium strains that are resistant to vancomycin contain potent FPR2 agonists, suggesting a potential role for FPR2 in antibiotic-resistant infections.

N-Formyl peptides are potent immunocyte activators and, once released in the circulation, they induce modulation of vascular tone by cellular mechanisms that are not completely understood. Wenceslaus et al. (Medical Hypotheses, Volume 81, Issue 4, October 2013, Pages 532-535) have observed that N-formyl peptides from bacterial (such as N-Formyl-Met-Leu-Phe Synonym: Chemotactic peptide, N-Formyl-L-methionyl-L-leucyl-L-phenylalanine, fMLF, fMLP (misnomer but widely used) and mitochondrial (such as formylated peptide corresponding to the NH₂-terminus of mitochondria NADPH dehydrogenase subunit 6; fMIT) sources induce FPR-mediated vasodilatation in resistance arteries. In general, both bacterially or mitochondrially derived as well as synthetic peptides that contain N-formyl-methionine are chemoattractants for phagocytic leukocytes (Proc. Natl. Acad. Sci. USA 72: 1059). In mammalian cells, cleavage products of mitochondrial proteins bearing N-formyl-methionine have also been shown to possess neutrophil chemotactic activity. The prototype formyl peptide, N-formyl-Met-Leu-Phe (fMLF), binds to human neutrophil receptors with high affinity. The formylated mitochondrial peptide receptors active on neutrophils belong to the formyl peptide receptor (FPR) family, which in humans constitutes FPR1, FPR2/ALX (lipoxin receptor) and FPR3. These are well conserved G protein-coupled receptors that have pluripotent and diverse roles in the initiation and resolution of inflammation. While FPR1 has relatively specific chemoattractant binding to only formylated peptides, Annexin A1 and Cathepsin G, FPR2 is a highly promiscuous receptor that can bind a variety of chemoattractant motifs in, for example, lipids, peptides and proteins to exert ligand-dependent pro-inflammatory or pro-resolution/anti-inflammatory effects. The role of FPR3, however, is less clear and likely plays only a subtle role in inflammation, although this still has to be fully elucidated. Another FPR-agonist is chemoattractant peptide WKYMVm (Trp-Lys-Tyr-Met-Val-D-Met (SEQ ID NO:6)), that seems to bind to FPR and FPRL1. Exactly how this peptide interacts with its receptors is unclear at present. It is intriguing that the human FPR interacts with both fMLF and WKYMVm (SEQ ID NO:6) efficiently, whereas the mouse FPR favors WKYMVm (SEQ ID NO:6) over fMLF. Furthermore, competitive binding assays with hFPR demonstrated that WKYMVm (SEQ ID NO:6), like fMLF, can displace [³H]fMLF bound to hFPR, suggesting that these two peptides interact with the human receptor using a similar mechanism or they may occupy the same or overlapping binding pocket.

CXC chemokine receptors are integral membrane proteins that specifically bind and respond to cytokines of the CXC chemokine family. Chemokine receptors (nomenclature agreed by NC-IUPHAR Subcommittee on Chemokine Receptors) comprise a large subfamily of GPCR activated by one or more of the chemokines, a large family of small cytokines typically possessing chemotactic activity for leukocytes. Chemokines can be divided by structure into four subclasses by the number and arrangement of conserved cysteines. CC (also known as β-chemokines), CXC (also known as α-chemokines and CX₃C chemokines all have four conserved cysteines, with zero, one and three amino acids separating the first two cysteines, respectively. C chemokines have only the second and fourth cysteines found in other chemokines. Chemokines can also be classified by function into homeostatic and inflammatory subgroups. Most chemokine receptors are able to bind multiple high affinity chemokine ligands, but the ligands for a given receptor are almost always restricted to the same structural subclass. Most chemokines bind to more than one receptor subtype. Receptors for inflammatory chemokines are typically highly promiscuous with regard to ligand specificity and may lack a selective endogenous ligand. There are currently six known CXC chemokine receptors in mammals, named CXCR1 through CXCR6, and several of these bind to a chemoattractant with motif Acetyl-proline-glycine-proline (AcPGP). The unacetylated chemoattractant peptide (PGP) also evokes neutrophil chemotaxis but is 4-7-fold less potent [J. Immunol. 2008; 180: 5662-5669].

Weathington et al. [Nat. Med. 2006; 12: 317-323.], convincingly demonstrated that PGP functioned as a neutrophil chemoattractant by mimicking key sequences found in classical neutrophil chemokines and signaling through CXCR1/2. Accordingly, intratracheal instillation of AcPGP dose dependently elicited neutrophilic inflammation in the airways of mice that was abolished in cxcr2−/− animals. Subsequent studies have highlighted downstream signaling events following CXCR1/2 engagement by PGP [PloS One 2011; 6: e1578, Eur. J. Pharmacol. 2011; 668: 428-434] and demonstrated the capacity of PGP to drive neutrophil superoxide production and matrix metalloproteinase (MMP)-9 and CXCL8 release. Typical peptide ligand chemoattractant motifs through which binding is achieved may comprise so-called PGP-peptides that arise from exposed stressed and damaged extracellular matrix collagens.

Most CXC chemokines active on neutrophils possess a Glu-Leu-Arg (ELR) motif and are identified as ELR-positive CXC chemokines. In humans, these include IL-8, active on CXCR1 and 2 chemokine receptors, and the GRO-α, β and γ chemokines, which ligate only CXCR2. Several ELR+ CXC chemokines contain a conserved PPGPH sequence (SEQ ID NO:7) immediately N-terminal to the third structural cysteine, while IL-8 has the sequence ESGPH (SEQ ID NO: 8) in this position. Structure-function studies of IL-8 show the “SGP” or “PGP” motif as a very important requirement for neutrophil cell binding and activation in radioligand and elastase release assays, respectively. Collagen represents in excess of 90% of the total protein mass of the extracellular matrix (ECM) in mammals, and proteolytic processing of native collagen can yield the tripeptide PGP. The prevalence of the PGP sequence within collagen molecules (28 PGP sequences per type I collagen fibril, 43 per type III collagen, 25 per type IV collagen and 44 per type V collagen) results in a potentially abundant bioactive signaling moiety that remains cryptic within the triple helical structure of collagen until liberated by proteolytic processing. Pfister et al. [Invest. Ophthalmol. Vis Sci., 1995; 36: 1306-1316] first identified N-terminal acetylated PGP (AcPGP) and N-terminal methylated PGP in an alkali eye injury model in rabbits, demonstrating their capacity to drive neutrophil recruitment and ensuing corneal ulceration [Invest. Ophthalmol. Vis Sci. 1999; 40: 2427-2429]. While conventional glutamic acid-leucine-arginine+(ELR+) CXC neutrophil chemokines are functional at nanomolar levels, AcPGP was subsequently demonstrated to operate at a micromolar level [Nat. Med. 2006; 12: 317-323], whereby AcPGP causes chemotaxis and production of superoxide through binding to CXC receptors, and administration of peptide causes recruitment of neutrophils (PMNs) into lungs of control, but not CXCR2-deficient mice. For N-acetyl Ser-Gly-Pro, (SGP) similar activity was found as that seen with N-acetyl Pro-Gly-Pro (PGP).

As documented above, in the art, finding functional equivalent chemoattractant motifs capable of inducing neutrophil chemotaxis is well developed. Also, it must be noted that many, if not all of the chemoattractant motifs shown herein induce cross-desensitization in neutrophils. Cross-desensitization is the heterologous desensitization of chemoattractant receptors; that is, stimulation of neutrophils with one chemoattractant renders the cells unresponsiveness to subsequent stimulation with (seemingly) unrelated other chemoattractants. Indeed, the stimulation of FPR1 with fMLF desensitized not only FPR1 but also C5aR and chemokine (C—X-C motif) receptor 2 (CXCR2), and inhibited neutrophil responses, such as calcium mobilization and chemotaxis, which are induced by C5a or IL-8 (Proc. Natl. Acad. Sci. U.S.A 1991 Dec. 15; 88(24):11564-8, J. Immunol. 1997 Feb. 1; 158(3):1361-9, J. Immunol. 1994 Oct. 1; 153(7):3267-75, J. Immunol. 1997 Mar. 1; 158(5):2340-9, J. Biol. Chem. 1993 Dec. 5; 268(34):25395-401. Furthermore, although it was initially thought that FPR only bound N-formylated peptides, it is now widely recognized that the formyl group is not a prerequisite for receptor binding. The N-formylated version of any peptide containing a methionine residue at the 5′ terminus is at least 100-fold more potent than the identical nonformylated peptide. However, if the peptide contains five or more amino acids, the non-formylated moieties can also bind and activate FPR (Pharmacol. Ther. 1997; 74:73-102). Modulation of FPR can also occur after activation of CD88 [a complement component 5a (C5a) receptor] or chemokine (C—X-C motif) receptor 2 CXCR2; (an IL-8 receptor) due to shared components of intracellular signaling molecules and occurs principally through protein kinase C-mediated pathways (J. Biol. Chem. 1999; 274:6027-6030). Similarly, WRWWWW (SEQ ID NO:9) is reported an analog of WKYMVm (SEQ ID NO:6) (Am. J. Pathol. 2015 May; 185(5): 1172-1184, see also Table 1 therein incorporated herein by reference). A divers panel of decapeptide agonists corresponding to the C-terminal “effector” region of human C5a (C5a65-74 or ISHKDMQLGR (SEQ ID NO:1)) is already discussed above. In short, many and widely variable neutrophil chemoattractants are known to interchangeably induce neutrophil chemotaxis, subsequently desensitize the neutrophil and induce clearance of debris through phagocytosis of tissue biomaterials such as peptides, lipids, glycoconjugates, nucleic acids, etcetera, contacting or surrounding the neutrophil.

Glutamine (Gln, Q) is the most abundant free amino acid in the plasma and tissue pool. It serves as an important fuel source for rapidly dividing cells, especially leucocytes and enterocytes. Glutamine is the most abundant nonessential amino acid in the body and in states of stress it becomes a conditionally essential amino acid. It is the preferred fuel source for the small bowel enterocyte, which is thought to help maintain its structure and function during times of stress. In septic and malnourished patients, muscle glutamine is depleted, and it is hypothesized that in these patients the availability of glutamine in lymphocytes and the gut is reduced, resulting in increased risk of sepsis. Although enteral formulas designed to improve immunity have given mixed results, glutamine supplementation has been shown not to be harmful and in fact reduced complications in patients with bone marrow transplantation, after surgery, and in patients with critical illness and severe burns.

Studies using parenteral glutamine have generally been more positive than those employing enteral glutamine. Although it is considered a non-essential amino acid, many studies showed that Gln has beneficial anti-inflammatory and tissue-regenerating properties and is considered conditionally essential for patients with catabolic conditions [J. Nutr. 131: 2543S-2549S discussion 2550S-2541S; Nutr. Rev. 48: 297-309. doi: 10.1111/j.1753-4887.1990.tb02967.x; Yonsei Med. J. 52: 892-897. doi: 10.3349/ymj.2011.52.6.892; Lancet 336: 523-525. doi: 10.1016/0140-6736(90)92083-t; PLoSONE 9(1): e84410.doi:10.1371/journal.pone.0084410]. Shiomi et al. [Inflamm. Bowel Dis. 17: 2261-2274. doi: 10.1002/ibd.21616] reported that Gln levels of serum and colon tissues were significantly lower in the acute phase of colonic inflammation, and Gln supplementation attenuated the degree of microscopic injury induced by dextran sulfate sodium (DSS). Also, glutamine and alanyl-glutamine dipeptide reduce vascular permeability with mesenteric plasma extravasation, leukocyte adhesion and tumor necrosis factor-α (TNF-α) release during experimental endotoxemia [Scheibe, Ricardo et al., 2009,—60 Suppl. 8 Journal of physiology and pharmacology: an official journal of the Polish Physiological Society].

Glutamine-containing di-peptides, such as alanyl-glutamine (in one-letter-code AQ, tradename DIPEPTIVIN®), glycyl-glutamine (GQ), leucyl-glutamine (LQ), valyl-glutamine (VQ), isoleucyl-glutamine (IQ), and cysteinyl-glutamine (CQ), have earlier been found useful in the treatment of various conditions (see also US2005/0059610 that discloses the use of glutamine to treat injury). Tri- and tetrapeptide formulations comprising glutamine (such as LQG, LQGV (SEQ ID NO:10), AQG, or AQGV (SEQ ID NO:11), see also WO2004/093897 or WO2012/112048) are, above the di-peptides listed, advantageously used in methods and pharmaceutical compositions to treat severe systemic inflammatory conditions.

Inflammatory diseases also involve autophagy, which is a broader phenomenon and covers many diseases. Indeed, the tri- and tetra-peptides are synthetic linear glutamine-containing peptides derived from the beta-human chorionic gonadotropin hormone, which have tissue-protective effects in animal studies, and have been shown to improve or therapeutically modulate vascular permeability, tissue repair and immune responses in human and non-human primates as well. WO2004093897 nor WO2012112048 are targeting specific (subsets) of cells.

For example, the tetrapeptide LQGV (SEQ ID NO:10) has been shown (van den Berg et al., Crit. Care Med 39: 126-134) to reduce mortality in a murine polymicrobial sepsis model. LQGV (SEQ ID NO:10) (at 5 mg/kg bodyweight) significantly improved survival from 20% to 50% during the first 5 days after moderate cecal ligation and puncture. This was associated with reduced cytokine and E-selectin levels in peritoneal lavage fluid, lungs, and, to a lesser extent, in plasma. LQGV (SEQ ID NO:10) treatment also reduced pulmonary nuclear factor-κB activation and pulmonary damage. In a severe cecal ligation and puncture model, the tetrapeptide LQGV (SEQ ID NO:10) (at 5 mg/kg bodyweight) combined with fluid resuscitation and antibiotics resulted in significantly better survival (70%) than that observed with fluid resuscitation and antibiotics alone (30%).

Also, the tetrapeptide AQGV (SEQ ID NO:11) has been shown (Gueller et al., PLoS One. 2015 Jan. 24; 10(1): e0115709. doi: 10.1371/journal.pone.0115709. eCollection 2015) to improve survival and attenuate loss of kidney function in mouse models renal ischemia/reperfusion injury (IRI) and of ischemia-induced delayed graft function after allogenic kidney transplantation. IRI was induced in male C57B1/6N mice by transient bilateral renal pedicle clamping for 35 minutes. Treatment with AQGV (SEQ ID NO:11) (20-50 mg/kg twice daily i.p. for four consecutive days) was initiated 24 hours after IRI when acute kidney injury (AKI) was already established. The treatment resulted in markedly improved survival in a dose dependent manner. Acute tubular injury two days after IRI was diminished and tubular epithelial cell proliferation was significantly enhanced by AQGV (SEQ ID NO:11) treatment. Furthermore, CTGF up-regulation, a marker of post-ischemic fibrosis, at four weeks after IRI was significantly less in AQGV (SEQ ID NO:11) treated renal tissue. Next, AQGV (SEQ ID NO:11) treatment was tested in a model of ischemia-induced delayed graft function after allogenic kidney transplantation. The recipients were treated with AQGV (SEQ ID NO:11) (50 mg/kg) twice daily i.p., which improved renal function and allograft survival by attenuating ischemic allograft damage.

The tetrapeptide AQGV (SEQ ID NO:11) has also been shown (Groenendael et al., Intensive Care Medicine Experimental 2016, 4 (Suppl. 1):A132) to be safe and have significant beneficial immunomodulatory effects in an experimental model of systemic inflammatory response syndrome (SIRS) in humans. SIRS can lead to pronounced tissue damage and is a frequent cause of multi-organ failure and mortality in intensive care units. SIRS can be elicited by a variety of insults, such as sepsis, trauma and major surgery, and no specific therapy is currently in routine use. To investigate the tolerability, safety and immunomodulatory effects of the tetrapeptide with AQGV (SEQ ID NO:11) in humans, a double blind, placebo controlled, dose-escalating randomized clinical trial in 60 healthy volunteers has been conducted. The study was carried out in two phases. In the first phase (n=24), safety and tolerability was established for escalating doses of the peptide (30, 90, and 180 mg/kg). In the second phase (n=36), the same doses were used to assess the effects of the peptide on systemic inflammation (SIRS) during experimental human endotoxemia. At t=0 hours, 2 ng/kg E. Coli endotoxin was administered i.v. followed by a 2-hour continuous i.v. infusion of tetrapeptide AQGV (SEQ ID NO:11) or placebo. Levels of circulating cytokines and adhesion molecules as well as body temperature and flu-like symptoms were assessed.

The tetrapeptide AQGV (SEQ ID NO:11) was well tolerated and showed an excellent safety profile. Treatment with at 180 mg/kg (the highest dose) of the peptide, but not with the lower doses tested, resulted in a significant attenuation of the endotoxin induced increase in plasma levels of IL-6, IL-8, IL-iRA, MCP-1, MIP-1α, and MIP-10 and the adhesion molecule VCAM-1. Furthermore, the highest dose reduced fever and flu-like symptoms. It was concluded that administration of the tetrapeptide AQGV (SEQ ID NO:11) is safe and results in attenuation of the systemic inflammatory response in humans. However, a drawback of the here above discussed di-, tri- and tetra-peptides for parenteral application is that they run the chance of being rapidly hydrolyzed in the blood before they have reached their target cells and can exert their beneficial actions. This rapid loss of peptide necessitates using high doses and long applications times to obtain the desired beneficial effects.

Furthermore, several publications (see, for example, WO2020/245299 or WO2010/108016) recognize benefits in potentiating musculoskeletal effects (for example, to treat frailty or cachexia in elderly) with compositions of individual or mixtures of individual anabolic amino acids (that activate mTOR and therewith inhibit autophagy) such as leucine, isoleucine, glutamine, and citrulline, wherein such compositions are preferably balanced or over-supplemented with one or more individual autophagy-inducing amino acids to achieve neutral net effects on autophagy as a whole. Where above applications do provide such compositions optionally as hydrolyzed proteins or peptides, such proteins or peptides have not been provided with means to target these compositions to desired cells. Meijer, A. J., Lorin, S., Blommaart, E. F. et al. Regulation of autophagy by amino acids and MTOR-dependent signal transduction, Amino Acids 47, 2037-2063 (2015), reviews the effects of amino acids, and foremost show that leucine (but not the other branched-chain amino acids), independent of the cell type, was the most effective amino acid in stimulating signaling mTOR but that, in addition, some other amino acids were required. In analogy with the inhibition of autophagy and the stimulation of S6 phosphorylation by amino acids, it was proposed that leucine, in combination with amino acid-induced cell swelling, would be sufficient to stimulate signaling. In line with this, others showed synergy between glutamine, a potent amino acid in promoting cell swelling, and leucine with regard to S6K phosphorylation in hepatocytes. In intestinal cells, with their rapid growth, in addition to glutamine and leucine, arginine has also been mentioned as an activator of mTOR signaling. In CHO cells, arginine also stimulated mTOR signaling albeit less effective than leucine. Although never considered, it is possible that the effect of arginine may be attributed, at least in part, to glutamate produced from arginine by the combined actions of arginase, ornithine aminotransferase and pyrroline 5-carboxylate dehydrogenase. Involvement of NO production from arginine can also not be excluded (Angcajas et al.). Angcajas et al. (Diversity of amino acid signaling pathways on autophagy regulation: A novel pathway for arginine; doi: 10.1016/J.BBRC.2014.01.117) outlines Arg-regulated autophagy seemingly different from mTOR activation. Kovics et al. (Inhibition of autophagic vacuole formation and protein degradation by amino acids in isolated hepatocytes. Exp. Cell Res. 1981 Jun.; 133(2):431-6, suggests to mix amino acids that inhibit protein degradation and lower autophagy. EP2490021A1 recognizes that peptides such as AQGV (SEQ ID NO:11), LQGV (SEQ ID NO:10), AQG, LQG, QGV, AQ, LQ, GV or QG are capable of modulating cell signaling via at least one Pattern Recognition Receptor (PRR) signaling pathway and/or G protein coupled receptor (GPCR) signaling pathway, and are useful in treating inflammation; an autophagy-inhibiting character of such peptides is not recognized in EP2490021A1. CN107501405 relates to a kind of cell autophagy to suppress polypeptide, and its amino acid sequence is LPDISLKDLQFLQSFCPSEVQ (SEQ ID NO:12) (purportedly derived from FIP200 albumen), which may be understood as an autophagy-inhibiting peptide for use in treatment of cancer. No other therapeutic options than cancer treatment are contemplated in CN107501405. Meijer at al., Angcajas et al., Kovics et al., EP2490021, nor CN107501405 mention targeted delivery of amino acids to cells. Such targeted delivery to desired cells has recently been discussed in WO2021/040526 that relates to means and methods for the treatment of diseases involving autophagy by cells, which process is involved in mechanisms of tissue repair, vascular permeability and immune responses. It provides methods and means to target the elastin receptor complex specifically and to provide molecules and compositions comprising a specific targeting agent as well as amino acid compositions that are involved in the pathway of autophagy and the diseases related thereto. It also relates to peptide-drug development, in particular, to (the improvement of) autophagy-inhibiting amino acid-containing peptides, herein also identified as autophagy-inhibiting-peptides (AIP), more in particular, glutamine-containing peptides and/or glutamine and other autophagy-modulating amino acid-containing compositions useful in the treatment of vascular and inflammatory conditions. It further relates to the improvement of glutamine peptides useful in the treatment of diabetic, vascular and/or inflammatory conditions. It provides a Q—ER peptide, comprising a synthetic peptide or functional analogue thereof provided with at least one PG-domain amino acid motif xGxxPG (SEQ ID NO:13) or functional equivalent thereof, the PG—domain motif allowing targeting of the peptide to the elastin receptor complex (ER), wherein at least one amino acid at position x is selected from the group of amino acids alanine (in one letter code: A), glutamine (Q), glycine (G), valine (V), leucine (L), isoleucine (I), proline (P) and arginine (R), the peptide comprising at least one glutamine (Q).

### BRIEF SUMMARY

The disclosure provides a method for lowering autophagy in a neutrophil cell, comprising targeting a neutrophil cell (shorthand neutrophil) having a receptor associated with its surface that is capable of binding to a chemotactic motif W, by providing the cell with a molecule containing the chemotactic motif H, whereby the molecule further comprises a source of autophagy-inhibiting amino acids selected from the group consisting of alanine (in one letter code: A), glutamine (Q), glycine (G), valine (V), leucine (L), isoleucine (I), proline (P) and arginine (R), preferably selected from the group consisting of A, Q, G, L and P, most preferably selected from the group consisting of A, Q, L and P, even more preferably for at least 50%, more preferably at least 70% selected from the group consisting of A, Q or L.

Neutrophils are the first white blood cells recruited to sites of inflammation or other tissue stress, in response to chemotactic cues produced by stressed tissue cells and tissue-resident immune cells such as macrophages. Neutrophils, therefore, comprise a large proportion of the early cellular infiltrate in inflamed or stressed tissues and are the major constituent of pus. As indicated already above, some amino acids inhibit autophagy more than others. The disclosure provides a molecule capable of targeting the neutrophils by employing the chemotactic cues, targeting the neutrophils therewith and providing those targeted neutrophils with amino acids that inhibit autophagy more than other amino acids do, in order to modulate the neutrophil response, preferably under circumstances of stressed tissue cells. Such a molecule as provided herein is preferably a peptide, preferable an autophagy-inhibiting-peptide (AIP), the molecule provided with or contains a neutrophil-chemotactic motif H, and the molecule, preferably a peptide, preferably an AIP, that is also provided with or contains a source of autophagy-inhibiting amino acids selected from the group of alanine (in one letter code: A), glutamine (Q), glycine (G), valine (V), leucine (L), isoleucine (I), proline (P) and arginine (R), preferably selected from the group A, Q, G, L and P, most preferably selected from the group A, Q, L and P, even more preferably selected from Q and L. The chemotactic motif W is preferably selected from the group of neutrophil-chemotactic motifs represented by fMLP, WKYMVm (SEQ ID NO:6), xPGP (SEQ ID NO:16), AcPGP, SGP, AcSGP, YSFKDMQLGR (SEQ ID NO:3) and AcYSFKPMPLaR (SEQ ID NO: 14). Herewith the disclosure provides methods and means to target the neutrophil specifically through targeting chemotactic motif W on the surface of the neutrophil that is preferably selected from the group of neutrophil-chemotactic motifs represented by motifs fMLP, WKYMVm (SEQ ID NO:6), xPGP (SEQ ID NO:16), AcPGP, SGP, AcSGP, YSFKDMQLGR (SEQ ID NO:3) and AcYSFKPMPLaR (SEQ ID NO:14) and to provide molecules and compositions, such as peptides containing such fMLP, WKYMVm (SEQ ID NO:6), xPGP (SEQ ID NO:16), AcPGP, SGP, AcSGP, YSFKDMQLGR (SEQ ID NO:3) and AcYSFKPMPLaR (SEQ ID NO:2), and comprising a specific targeting agent as well as autophagy-inhibiting-amino acid compositions that are involved in the pathway of autophagy and the diseases related thereto. It also relates to peptide-drug development, in particular, to (the improvement of) autophagy-inhibiting amino acid-containing peptides, herein also identified as autophagy-inhibiting-peptides (AIP), more in particular, glutamine-containing peptides and/or glutamine and other autophagy-modulating amino acid-containing compositions useful in the treatment of vascular and inflammatory conditions. It further relates to the improvement of glutamine peptides useful in the treatment of diabetic, vascular and/or inflammatory conditions. It provides an AIP, comprising a synthetic peptide or functional analogue thereof provided with or containing at least one chemotactic amino acid motif W, preferably a motif selected from the group of motifs represented by motifs fMLP, WKYMVm (SEQ ID NO:6), xPGP (SEQ ID NO:16), AcPGP, SGP, AcSGP, YSFKDMQLGR (SEQ ID NO:3) and AcYSFKPMPLaR (SEQ ID NO:2) or functional equivalent thereof, motif allowing targeting of the peptide to the neutrophil wherein at least one amino acid is selected from the group of amino acids alanine (in one letter code: A), glutamine (Q), glycine (G), valine (V), leucine (L), isoleucine (I), proline (P) and arginine (R), the peptide preferably provided with at least one glutamine (Q).

Broadly, autophagy is important for immature neutrophil differentiation and mature function. Several studies suggest a dual role for autophagy in neutrophil function during inflammation. Augmentation of autophagy may be an effective target for enhancement of proper myeloid differentiation and antimicrobial defense, inducing increased NET formation, degranulation and inflammatory cytokine release. Conversely, autophagy inhibition may be useful in neutrophil-mediated inflammatory disease (Korean J. Physiol. Pharmacol. 2020 Jan.; 24(1): 1-10). Inhibition of autophagy reverses autophagic neutrophil death and slows disease development [Oncotarget. 2017 Sep. 26; 8(43):74720-74735]. Inhibition of autophagy during neutrophil-mediated inflammation and autoimmune disease reduced disease severity and progression by suppressing degranulation and ROS production [PLoS One. 2012; 7(12):e51727, ncbi.nlm.nih.gov/pmc/articles/PMC6940497/—B35 Nature. 2015 Dec. 24; 528(7583):565-9.]. Similarly, suppression of autophagy through NLRP3 knockdown or inhibition of the NLRP3 inflammasome enhanced neutrophil recruitment and phagocytosis, thereby improving bacterial clearance and augmenting the survival of septic mice [J. Immunol. 2017 Feb. 1; 198(3):1253-1262]. Depending on the inflammatory environment, autophagy in neutrophil may behave as a double-edged sword. While it is beneficial in combating infection, it may favor excessive inflammation through exaggerated NETs formation and cytokine release. Thus, autophagic homeostasis is important for proper neutrophil effector function and host health.

However, therapeutically targeting neutrophils have traditionally been considered to cause collateral tissue damage; and recent studies indicate a clear protective role for neutrophils during resolution and repair. The disclosure provides a method to exploit the broadly varied chemotactic and phagocytic repertoire of leukocytes, preferably of neutrophils, and bend their tissue damage potential toward more beneficial and therapeutic mechanisms of resolution and repair. The disclosure provides such a therapeutical approach by presenting such cells with a source of amino acids having the desired beneficial effects through cell-receptor-specific targeting of peptides carrying both a neutrophil-chemoattractant or chemotactic motif W, selected from the various motifs as discussed above. Such peptides according to the disclosure are limited in length and can be made with various tools and methods known in the art, such as by using a, preferably automated peptide synthesizer. Such a peptide according to the disclosure is obtainable or can be derived at with a peptide synthesis method as provided herein for use in a method selected from the group of lowering autophagy, modulating inflammation, in particular, by lowering NET formation, and/or degranulation and/or inflammatory cytokine release, modifying vascular permeability, improving tissue repair and modulating an immune response. Such an AIP according to the disclosure is particularly useful in reducing post-operative complications. It is preferred that the cell-receptor-specific targeting peptides are at least partly composed of autophagy-inhibiting amino acids. It is preferred that such targeted receptors are involved in receptor-induced neutrophil chemotaxis.

According to the present disclosure the peptides with III and with amino acids that have the beneficial effects are targeted to the cells in which they can have their beneficial effects, in particular, by targeting with a chemotactic motif W through any specific means. Preferably the targeting means enables internalization (i.e., by phagocytosis or endocytosis) of the beneficial amino acids and when the amino acids are provided in an oligopeptide format the internalization typically results in the oligopeptide being delivered to a lysosome (generally, and herein, also called autophagosome).

The disclosure relates to a distinct and new class of drugs: autophagy-inhibiting compounds that comprise peptides and/or amino acids that target the nutrient sensing system of the mechanistic target of rapamycin, mTOR and inhibit autophagy. Upon testing formyl-peptide related signaling effects of an autophagy-inhibiting AQGV (SEQ ID NO:11) peptide the peptide was found to unexpectedly attenuate p38/p38-MK2-HSP27 and/or PI3K/AKT/mTOR pathways that govern signal cytoskeleton contraction in modulating vascular permeability. Hence, the current disclosure relates to the targeted use of an autophagy-inhibiting peptide herein, for improving or modulating vascular permeability, tissue repair and immune responses and therapeutic uses thereof.

The disclosure provides a method for lowering autophagy, comprising targeting cells having a receptor associated with their surface that is capable of binding to a chemotactic motif of a molecule, with a molecule specifically recognizing the receptor, whereby the molecule, such as a peptide, is provided with a source of autophagy-inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). The targeting then results in delivering the source, as a package of autophagy-inhibiting amino acids, preferably a peptide, to the cell, where the molecule provided with the source or package is, for example, taken up by common endocytosis and/or phagocytosis, and then hydrolyzed into its collection of constituent, preferably, autophagy-inhibiting, amino acids in lysosomes (autophagosomes), and individual amino acids are released in the cytosol of the cell. In this way, the mechanistic target of rapamycin (mTOR) is activated by the collection of autophagy-inhibiting amino acid in the package selected for targeting of the peptide to the cell.

It is preferred that the molecule, preferably the AIP, comprises, more preferably consists of, a peptide comprising at least 4 amino acids and at most 30 amino acids comprising a sequence of the formula (φnW, or Wφn, or φWφm wherein W herein represents at least one of many a chemotactic or chemoattractant motifs as discussed above, φ is an autophagy-inhibiting amino acid, preferably selected from the group consisting of alanine (in one letter code: A), glutamine (Q), glycine (G), valine (V), leucine (L), isoleucine (I), proline (P) and arginine (R), more preferably selected from the group consisting of leucine (L), alanine (A), glutamine (Q), glycine (G) and proline (P), most preferably selected from the group consisting of leucine (L), alanine (A), glutamine (Q), and proline (P) and wherein n=an integer from 1 to 24 and m is an integer from 1-23, whereby n+m is no greater than 24, n and m denoting the number of consecutive p preceding or following W. In a preferred embodiment, n+m is no greater than 16, more preferably no greater than 12, more preferably no greater than 8. It is preferred that the receptor specifically recognizing the chemotactic motif W is selected from the group of formyl-peptide receptors, complement receptors and CXC-receptors, in particular, wherein the chemotactic motif W is selected from the group of chemoattractant motifs represented by fMLF, WKYMVm (SEQ ID NO:6), PGP, AcPGP, SGP, AcSGP, YSFKDMQLGR (SEQ ID NO:3) and AcYSFKPMPLaR (SEQ ID NO:2) (Ac herein denotes Acetyl-) and functionally equivalent chemoattractant peptide motifs H. Several of such AIPs are identified in detail in the description herein, others may be easily designed and obtained or derived at with means (such as synthesizing means) and methods (such as synthesizing methods) known in the art and arrive at an AIP according to the disclosure. In the art and identified herein, several inherently autophagy-inhibiting amino acids are known that may be used in an AIP as source of autophagy-inhibiting amino acids. Most preferred are Q and L, which can be used together or be interchanged for each other in several preferred AIPs. In the art, finding functional equivalent chemoattractant motifs (herein represented by I [inverted M] is well developed. Also, it must be noted that many, if not all of the chemoattractant motifs shown herein induce cross-desensitization in neutrophils, and can be used interchangeably as mutual alternatives of chemoattractant peptide with motif W when targeting a cell as provided herein with a molecule according to the disclosure. Cross-desensitization is the heterologous desensitization of chemoattractant receptors; that is, stimulation of neutrophils with one chemoattractant renders the cells unresponsiveness to subsequent stimulation with (seemingly) unrelated other chemoattractants, and chemoattractants that desensitize, for example, formyl-peptide receptors, complement receptors and/or CXC-receptors, or functional equivalents thereof, are herein grouped under chemoattractants with motif W.

In a preferred embodiment, the disclosure provides a method wherein the molecule with motif W comprises or is provided with or contains a peptide according to the disclosure wherein φn and/or φm comprise a dipeptide selected from the group AQ, LQ, PQ, VQ, GQ, a tripeptide selected from the group consisting of AQL, LQL, PQL, VQL, GQL, PLQ, LQG, PQV, VGQ, LQP, LQV, AQG, QPL, PQV, VGQ and GQG, or a tetrapeptide selected from LQGV (SEQ ID NO:10) and AQGV (SEQ ID NO:11), preferably selected from the group consisting of AQ, LQ, GQ, AQL, LQL, GQL, PLQ, LQG and AQG. It is preferred that the molecule is connected to the peptide through a peptide bond. The disclosure also provides a method for producing autophagy-inhibiting-peptide according to the disclosure comprising synthesizing the peptide with an automated peptide synthesizer, and provides a neutrophil-targeted autophagy-inhibiting peptide obtainable by synthesizing with an automated peptide synthesizer and use of the peptide for lowering autophagy of cells of a subject, in particular, when the subject is deemed to be in need of such treatment.

There is a certain balance to be achieved in the size of the oligopeptides provided by the disclosure. For speed of uptake and lower risk of immune responses smaller sizes are preferred, for half-life reasons and amount of autophagy lowering amino acids delivered longer sequences are preferred. Depending on the condition to be treated and the doses considered acceptable the skilled person will be capable of determining the right size of the oligopeptide or combinations of different sizes, optionally with additional autophagy lowering amino acids provided concomitantly (e.g., through conjugation to vehicles comprising the additional amino acids).

Therewith, the disclosure provides a method to regulate central cellular events that involve the mechanistic target of rapamycin (mTOR) pathway (Liu and Sabatini, Nature Reviews Molecular Cell Biology, volume 21, pages 183-203(2020)) The mTOR pathway integrates a diverse set of environmental cues, such as growth factor signals and nutritional status, to direct eukaryotic cell growth. Over the past two and a half decades, mapping of the mTOR signaling landscape has revealed that mTOR controls biomass accumulation and metabolism by modulating key cellular processes, including protein synthesis and autophagy, balancing mTOR activated proteogenesis versus proteolytic autophagy in a cell, respectively. The disclosure provides delivering a source of autophagy-inhibiting amino acids to a targeted cell, after which the cell and, in particular, the lysosomal compartment of the cell, is provided with the source of autophagy inhibition amino acids through endocytosis or phagocytosis and amino acids are liberated (e.g., through enzymatic hydrolysis) in the compartment and become available for cytosolic routing.

Given mTOR's pathway central role in maintaining cellular and physiological homeostasis, dysregulation of mTOR signaling has been implicated in many disorders with a general cellular origin, such as metabolic disorders as diabetes, neurodegeneration, cancer, inflammation and ageing. In particular, the mechanistic target of rapamycin complex I (mTORC1) is a central regulator of cellular and organismal growth, and this pathway is implicated in the pathogenesis of many animal- and human diseases. mTORC1 promotes growth in response to the availability of nutrients, such as amino acids in lysosome and transferred to cytosol, which drive mTORC1 to the lysosomal surface, its site of activation. According to the disclosure, some amino acids activate mTOR more than others, and therewith inhibit autophagy or stimulate proteogenesis more than others, in particular, amino acids selected from the group consisting of alanine (in one letter code: A), glutamine (Q), glycine (G), valine (V), leucine (L), isoleucine (I), proline (P) and arginine (R) are known to inhibit autophagy more than other natural occurring amino acids. The disclosure now provides targeting collections or strings of such selected autophagy-inhibiting amino acids delivered at cells, such as neutrophils, that help an organism tackle or combat disease by curing tissue defects central to the health of an organism, in particular, of a human organism; the cells in and around the vascular system that are central in curative activity and relate to vascular integrity or permeability, to tissue repair and to innate and adaptive immune responses, all cells that, in various ways, are involved in curing an organism from damage resulting from insult, injury, infection, metabolic alteration and cellular degeneration.

Central to the delivery of such sources of autophagy-inhibiting amino acids to cells and tissues in curative need, the disclosure provides use of targeted delivery of a collection or source of such amino acids to cells having a receptor associated with their surface that is capable of binding to a chemotactic motif of a molecule, as these cells (examples of cells having or carrying a surface-associated CXC receptor in at least a part of their life cycle are neutrophils) are typically involved in curative activities that benefit from lowered and at least partly inhibited autophagy and likewise increased and improved mTOR-mediated proteogenesis.

In a preferred embodiment, the disclosure therewith provides a method for modifying vascular permeability, comprising targeting cells having a receptor associated with their surface that is capable of binding to a chemotactic motif of a molecule, with a molecule specifically recognizing the receptor, whereby the molecule is provided with a source of autophagy-inhibiting amino acids, selected from the group consisting of alanine (in one letter code: A), glutamine (Q), glycine (G), valine (V), leucine (L), isoleucine (I), proline (P) and arginine (R), more preferably selected from the group consisting of leucine (L), alanine (A), glutamine (Q), glycine (G) and proline (P). Increased vascular permeability is, for example, governing fluid and white blood cell (neutrophil) extravasation, that is initially required in acute inflammations, but that in a later stage typically needs inhibition or reduction (i.e., lower permeability, or return to original vascular integrity) to allow for repair of tissue after, for example, inflammation has had its function and tissue is set to regain its integrity and be healed.

Therewith, the disclosure is also providing a method for improving or promoting tissue repair, comprising targeting cells having a receptor associated with their surface that is capable of binding to a chemotactic motif of a molecule, with a molecule specifically recognizing the receptor, whereby the molecule is provided with a source of autophagy-inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P), most preferably selected from the group leucine (L), alanine (A), glutamine (Q), and proline (P).

Restoring tissue integrity is particularly beneficial when acute immune responses need to be dampened and to switch the immune response toward a curative and tissue repairing response. The disclosure therewith provides a method for modulating an immune response, comprising targeting cells having a receptor associated with their surface that is capable of binding to a chemotactic motif of a molecule, with a molecule specifically recognizing the receptor, whereby the molecule is provided with a source of autophagy-inhibiting amino acids, selected from the group consisting of alanine (in one letter code: A), glutamine (Q), glycine (G), valine (V), leucine (L), isoleucine (I), proline (P) and arginine (R), more preferably selected from the group consisting of leucine (L), alanine (A), glutamine (Q), glycine (G) and proline (P), most preferably selected from the group consisting of leucine (L), alanine (A), glutamine (Q), and proline (P).

It is preferred that the source of autophagy-inhibiting amino acids is a peptide comprising the autophagy-inhibiting amino acids. For such purpose, it is preferred that the chemoattractant motif W comprising autophagy-inhibiting peptide comprising the autophagy-inhibiting amino acids comprises a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), preferably selected from the group AQ, LQ, GQ, AQL, LQL, GQL, PLQ, LQG, AQG. It is moreover preferred that the chemoattractant motif W is connected to the peptide comprising the autophagy-inhibiting amino acids by a peptide bond. These molecules of the disclosure can be simply produced by peptide synthesizers and can be readily degraded once in the lysosome to produce the autophagy-inhibiting amino acids.

The disclosure also provides a molecule specifically recognizing a CXC receptor for use in lowering autophagy, the molecule comprising a source of autophagy-inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). Preferably, such a molecule as provided herein comprises at least 4 amino acids and at most 30 amino acids comprising a sequence of the formula φnPG, or PGφn, or φPGφm wherein φ is an autophagy-inhibiting amino acid and n=an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a chemoattractant motif W comprising autophagy-inhibiting peptide according to the disclosure wherein φn and/or φm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), preferably selected from the group AQ, LQ, GQ, AQL, LQL, GQL, PLQ, LQG, AQG.

The disclosure also provides a molecule specifically recognizing a CXC receptor for use in the modulation of an immune response, the molecule comprising a source of autophagy-inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). Preferably, such as molecule as provided herein comprises a peptide (herein also termed chemoattractant motif W comprising autophagy-inhibiting peptide) comprising at least 4 amino acids and at most 30 amino acids comprising a sequence of the formula φnPGP, or PGPφn, or φPGPφm wherein x is a naturally occurring amino acid, φ is an autophagy-inhibiting amino acid and n=an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a chemoattractant motif W comprising autophagy-inhibiting peptide according to the disclosure wherein φn and/or φm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), preferably selected from the group AQ, LQ, GQ, AQL, LQL, GQL, PLQ, LQG, AQG.

The disclosure also provides a molecule specifically recognizing a CXC receptor for use in improving tissue repair, the molecule comprising a source of autophagy-inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). Preferably, such as molecule as provided herein comprises a peptide (herein also termed chemoattractant motif WII comprising autophagy-inhibiting peptide) comprising at least 4 amino acids and at most 30 amino acids comprising a sequence of the formula φnPGP, or PGPφn, or φPGPφm φ is an autophagy-inhibiting amino acid and n=an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a chemoattractant motif W comprising autophagy-inhibiting peptide according to the disclosure wherein φn and/or φm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), preferably selected from the group AQ, LQ, GQ, AQL, LQL, GQL, PLQ, LQG, AQG.

The disclosure also provides a molecule specifically recognizing a CXC receptor for use in modifying vascular permeability, the molecule comprising a source of autophagy-inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). Preferably, such as molecule as provided herein comprises a peptide (herein also termed chemoattractant motif W comprising autophagy-inhibiting peptide) comprising at least 4 amino acids and at most 30 amino acids comprising a sequence of the formula φnPGP, or PGPφn, or φPGPφm wherein x is a naturally occurring amino acid, φ is an autophagy-inhibiting amino acid and n=an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a chemoattractant motif W comprising autophagy-inhibiting peptide according to the disclosure wherein φn and/or φm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), preferably selected from the group AQ, LQ, GQ, AQL, LQL, GQL, PLQ, LQG, AQG.

Moreover, the disclosure also provides alternative modes of targeting cells having a receptor associated with their surface that is capable of binding to a chemotactic motif of a molecule, with a molecule specifically recognizing the receptor, whereby the molecule is provided with a source of autophagy-inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P).

In one further embodiment, the disclosure provides a method for lowering autophagy, comprising targeting cells having a receptor associated with their surface that is capable of binding to a chemotactic motif of a molecule, with a molecule specifically recognizing the receptor, wherein the molecule recognizing the receptor is an antibody-like molecule, preferably selected from IgG, IgM, single chain antibodies, FAB- or FAB′2-fragments. In principle any antibody-like molecule that can specifically recognize a CXC receptor may be used, whereby single chain formats (one polypeptide chain only) including at least one Vh or Vhh are preferred. These formats are preferred because they can be used as oligopeptide with the autophagy lowering amino acids bound to them through peptide bonds. Antibody-like molecules are general rapidly phagocytosed upon binding to their target and delivered in the lysosomal compartment, where the amino acid of that source can be further utilized for mTOR activation. Thus, internalizing antibody-like molecules are preferred. It is preferred that the source of autophagy-inhibiting amino acids is a peptide comprising a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), connected to the antibody-like molecule through a peptide bond; alternatively, the antibody-like molecule is otherwise conjugated to the source of autophagy-inhibiting amino acids. Conjugation methods are known in the art. Likewise, the source of autophagy-inhibiting amino acids is a lipid vesicle such as a liposome, in particular, wherein the liposome comprises a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), preferably selected from the group AQ, LQ, GQ, AQL, LQL, GQL, PLQ, LQG, AQG.

The disclosure provides a synthetic glutamine peptide that has been provided with a chemotactic motif and also provided (enriched) with selected amino acids that preferentially inhibit (mTOR-mediated) autophagy of a cell after the peptide is hydrolyzed into its individual amino acid components in the lysosome of the cell. Inhibiting autophagy, by these selected autophagy-inhibiting amino acids, modulates the activity of immune cells; inhibiting autophagy, by these selected autophagy-inhibiting amino acids, modulates the permeability of vascular cells. These actions, alone or combined, contribute to the immune and/or vascular cells showing curative tissue repair activities after having been targeted with a peptide according to the disclosure. The disclosure provides a curative and tissue repair supporting chemoattractant motif W comprising autophagy-inhibiting peptide, the peptide comprising a synthetic peptide or functional analogue thereof, provided with a glutamine (Q) and with a CXC-receptor binding amino acid sequence motif and also comprising at least 50% amino acids selected from the group of autophagy-inhibiting amino acids alanine (in one letter code: A), glutamine (Q), glycine (G), valine (V), leucine (L), proline (P), and arginine (R).

In a preferred embodiment, the disclosure provides a chemoattractant motif Wll comprising autophagy-inhibiting peptide or functional analogue, that has been provided with a chemotactic motif and also comprises at least 60%, more preferably at least 75%, most preferably at least 90% amino acids selected from the group of autophagy-inhibiting amino acids alanine (in one letter code: A), glutamine (Q), glycine (G), valine (V), leucine (L), proline (P), and arginine (R).

Such a peptide is herein also called a chemoattractant motif W comprising autophagy-inhibiting peptide. In a first embodiment, a chemoattractant motif W comprising autophagy-inhibiting peptide, comprising or consisting of a synthetic peptide or functional analogue thereof, is provided with at least one CXC-receptor binding amino acid motif, such as a PGP-domain amino acid motif PGP or PGP or functional equivalent thereof, the PGP-domain motif allowing targeting of the peptide to the CXC receptor, wherein at least one amino acid at position x is selected from the group of amino acids alanine (in one letter code: A), glutamine (Q), glycine (G), valine (V), leucine (L), isoleucine (I), proline (P) and arginine (R), the peptide provided with at least one glutamine (Q).

In a preferred embodiment, the disclosure also provides a peptide (herein also termed chemoattractant motif W comprising autophagy-inhibiting peptide) comprising at least 4 amino acids and at most 30 amino acids comprising a sequence of the formula φnPGP, or PGPφn, or φPGPφm wherein x is a naturally occurring amino acid, φ is an autophagy-inhibiting amino acid and n=an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a chemoattractant motif W comprising autophagy-inhibiting peptide according to the disclosure wherein φn and/or φm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), preferably selected from the group AQ, LQ, GQ, AQL, LQL, GQL, PLQ, LQG, AQG.

The disclosure also provides a pharmaceutical formulation comprising a peptide (herein also termed chemoattractant motif W comprising autophagy-inhibiting peptide) comprising at least 4 amino acids and at most 30 amino acids comprising a sequence of the formula φnPGP, or PGPφn, or φPGPφm wherein x is a naturally occurring amino acid, φ is an autophagy-inhibiting amino acid and n=an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a pharmaceutical formulation comprising a peptide comprising xPGPx (SEQ ID NO:18) and a peptide selected from dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), for use in lowering autophagy, wherein x represents no amino acid or a naturally occurring amino acid and at least one pharmaceutically acceptable excipient.

The disclosure also provides a pharmaceutical formulation comprising a peptide (herein also termed chemoattractant motif WW comprising autophagy-inhibiting peptide) comprising at least 4 amino acids and at most 30 amino acids comprising a sequence of the formula φnPGP, or PGPφn, or φPGPφm wherein x is a naturally occurring amino acid, φ is an autophagy-inhibiting amino acid and n=an integer from 1 to 24 and m is an integer from 1-23, whereby n+m is no greater than 24. In a preferred embodiment, n+m is no greater than 16, more preferably no greater than 12, more preferably no greater than 8, and at least one pharmaceutically acceptable excipient. In a preferred embodiment, the disclosure provides a pharmaceutical formulation comprising a peptide comprising xPGPx (SEQ ID NO:18) and a peptide selected from dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), for use in modifying vascular permeability, wherein x represents no amino acid or a naturally occurring amino acid and at least one pharmaceutically acceptable excipient.

The disclosure also provides a pharmaceutical formulation comprising a peptide (herein also termed chemoattractant motif WW comprising autophagy-inhibiting peptide) comprising at least 4 amino acids and at most 30 amino acids comprising a sequence of the formula φnPGP, or PGPφn, or φPGPφm wherein x is a naturally occurring amino acid, φ is an autophagy-inhibiting amino acid and n=an integer from 1 to 24 and m is an integer from 1-23, whereby n+m is no greater than 24. In a preferred embodiment, n+m is no greater than 16, more preferably no greater than 12, more preferably no greater than 8, and at least one pharmaceutically acceptable excipient. In a preferred embodiment, the disclosure provides a pharmaceutical formulation comprising a peptide comprising xPGPx (SEQ ID NO:18) and a peptide selected from dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), for use in modulating an immune response, wherein x represents no amino acid or a naturally occurring amino acid and at least one pharmaceutically acceptable excipient.

The disclosure also provides a pharmaceutical formulation comprising a peptide (herein also termed chemoattractant motif W comprising autophagy-inhibiting peptide) comprising at least 4 amino acids and at most 30 amino acids comprising a sequence of the formula φnPGP, or PGPφn, or φPGPφm wherein x is a naturally occurring amino acid, φ is an autophagy-inhibiting amino acid and n=an integer from 1 to 24 and m is an integer from 1-23, whereby n+m is no greater than 24. In a preferred embodiment, n+m is no greater than 16, more preferably no greater than 12, more preferably no greater than 8 and at least one pharmaceutically acceptable excipient. In a preferred embodiment, the disclosure provides a pharmaceutical formulation comprising a peptide comprising xPGPx (SEQ ID NO:18) and a peptide selected from dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), for use in improving or promoting tissue repair, wherein x represents no amino acid or a naturally occurring amino acid and at least one pharmaceutically acceptable excipient.

The disclosure also provides a pharmaceutical formulation comprising a peptide (herein also termed chemoattractant motif W comprising autophagy-inhibiting peptide) comprising at least 4 amino acids and at most 30 amino acids comprising a sequence of the formula φnPGP, or PGPφn, or φPGPφm wherein x is a naturally occurring amino acid, φ is an autophagy-inhibiting amino acid and n=an integer from 1 to 24 and m is an integer from 1-23, whereby n+m is no greater than 24. In a preferred embodiment, n+m is no greater than 16, more preferably no greater than 12, more preferably no greater than 8, further comprising insulin, preferably for use in the treatment of impairment of pancreatic beta-cell function. In a preferred embodiment, the disclosure provides a pharmaceutical formulation comprising a peptide comprising xPGPx (SEQ ID NO:18) and a peptide selected from dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO: 11), for use in lowering autophagy, wherein x represents a naturally occurring amino acid, further comprising insulin, preferably for use in the treatment of impairment of pancreatic beta-cell function.

Typically, the pharmaceutical formulations of the disclosure are intended for parenteral administration. In animal studies and clinical studies safe and efficacious doses can be established according to dose finding protocols well known to the skilled person. Typically, peptides according to the disclosure without any targeting means need to be provided at high doses because of the limited half-life of oligopeptides in circulation. It is one of the advantages of the present disclosure that by targeting less random circulation will occur and that by targeting more amino acids of the disclosure will be delivered where needed and, therefore, doses may be lower than of the peptides without targeting.

The disclosure further relates to the improvement of peptides comprising glutamine (Q), allowing efficient targeting of the glutamine-containing peptide (herein also termed glutamine peptide) to cells where the chemoattractant motif W comprising autophagy-inhibiting peptide can exert its effects, therewith improving dosing requirements. Such a chemoattractant motif W comprising autophagy-inhibiting peptide as provided by the disclosure is useful in methods and pharmaceutical compositions for the treatment of inflammatory and vascular conditions. The disclosure provides a synthetic chemoattractant motif W comprising autophagy-inhibiting peptide of at most 30 amino acids, preferably at most 20 amino acids, more preferably at most 15 amino acids, most preferably at most 9 amino acids, the motif allowing targeting of the chemoattractant motif W comprising autophagy-inhibiting peptide to the human CXC receptor (ER). Functional analogues of a chemoattractant motif W comprising autophagy-inhibiting peptide may be selected from peptides comprising amino acids selected from the group of amino acids alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). In a preferred embodiment, the disclosure provides for a chemoattractant motif H comprising autophagy-inhibiting peptide or functional analogue, that comprises at least 50%, more preferably at least 75%, most preferably at least 100% amino acids selected from the group of autophagy-inhibiting amino acids alanine (in one letter code: A), glutamine (Q), glycine (G), valine (V), leucine (L), proline (P), and arginine (R). In a more preferred embodiment, the disclosure provides for a chemoattractant motif W comprising autophagy-inhibiting peptide functional analogue, that comprises at least 50%, more preferably at least 75%, most preferably at least 100% amino acids selected from the group of autophagy-inhibiting amino acids alanine (in one letter code: A), glutamine (Q), glycine (G), valine (V), leucine (L), and proline (P). In a most preferred embodiment, the disclosure provides for a chemoattractant motif W comprising autophagy-inhibiting peptide functional analogue, that comprises at least 50%, more preferably at least 75%, most preferably at least 100% amino acids selected from the group of autophagy-inhibiting amino acids alanine (in one letter code: A), glutamine (Q), glycine (G), leucine (L), and proline (P). These amino acids and, in particular, glutamine (Q) and leucine (L), were shown to be most prominently capable of inhibiting mTOR-mediated autophagy, mTOR being an important switch governing proteogenesis and proteolysis (autophagy) in a cell.

Preferably, a functional analogue of the chemoattractant motif W comprising autophagy-inhibiting peptide has a length in the range of 4-12 amino acids, more preferably 6-12 amino acids. Preferably, such a functional analogue is a linear peptide. A functional chemoattractant motif W comprising autophagy-inhibiting peptide analogue according to the disclosure may be more preferably selected from the group consisting of peptides comprising a dipeptide sequence selected from the group of AQ, LQ, PQ, VQ, GQ. A functional chemoattractant motif W comprising autophagy-inhibiting peptide analogue according to the disclosure may be more preferably selected from the group consisting of peptides comprising a tripeptide sequence selected from the group of AQL, LQL, PQL, VQL, GQL, PLQ, LQG, PQV, VGQ, LQP, LQV, AQG, QPL, PQV, VGQ, GQG.

Herewith, the disclosure provides improved synthetic chemoattractant motif W comprising autophagy-inhibiting peptides (AIPs) for use in the treatment of a diabetic, inflammatory or vascular condition, preferably for the treatment of such a condition in a human, the chemoattractant motif W comprising AIP having been provided with a key motif of amino acids (PG-domain) allowing targeting to and docking of the improved chemoattractant motif W comprising autophagy-inhibiting peptide with cells carrying the human CXC receptor, a receptor complex involved in modulating immune cell reactivity and/or vascular cell repair. The beneficial anti-diabetic, anti-inflammatory and vascular repair effect of the chemoattractant motif W comprising AIP, once it has entered the target cell, is thought to be generated by inhibition of autophagy of the target cell through autophagy-inhibiting-amino acids generated by hydrolysis of the chemoattractant motif W comprising autophagy-inhibiting peptide and act on the mammalian target of rapamycin (mTOR) complex.

A preferred autophagy-inhibiting amino acid included in a chemoattractant motif W comprising autophagy-inhibiting peptide as provided by the disclosure is selected from the group of amino acids alanine (A), proline (P), leucine (L) and glutamine (Q). Most preferred autophagy-inhibiting amino acids for inclusion in a PG-domain comprising chemoattractant motif W comprising autophagy-inhibiting peptide according to the disclosure are L-leucine, L-glutamine and L-alanine.

Inhibition of autophagy in the target cells (cells with an active CXC receptor such as immune cells or vascular cells are preferably targeted) by a chemoattractant motif W comprising autophagy-inhibiting peptide according to the disclosure generally results in improved resistance to permeability and improved proliferation of vascular cells and reduced acute inflammatory activity and reduced extravasation of immune cells. Acute systemic conditions such as sepsis or systemic inflammatory response syndrome (SIRS), as well as chronic systemic vasculopathies in patients with a relative or absolute lack of C-peptide (as typically seen in type 1 diabetes and end-phase type 2 diabetes) often lead to a pathogenesis of micro-vascular damage involving detrimental activation and extravasation of immune cells (e.g., neutrophils, macrophages and lymphocytes) and destruction of the vascular cells (e.g., vascular endothelial cells, smooth muscle cells and fibroblasts), that form blood vessels. Targeting a chemoattractant motif W comprising autophagy-inhibiting peptide according to the disclosure to these cells where it is then hydrolyzed and inhibits autophagy through the action of its individual amino acids inhibiting autophagy allows reduction of these pathogenic events with beneficial effects to the treatment of a patient suffering from the diabetic, conditions often seen due to lack of C-peptide, and seen with other (micro-)vascular and/or inflammatory conditions.

Additional useful synthetic chemoattractant motif W comprising AIPs having been provided with a PG-domain binding motif are listed in the detailed description or elsewhere herein.

The peptides as provided herein are useful in the treatment of acute conditions, such as acute kidney injury, also in acute systemic inflammatory conditions such as sepsis or systemic inflammatory response syndrome (SIRS), leading to vascular damage and often aggravated by (multiple organ) organ failure, or inflammatory conditions. The peptides of the disclosure are particularly useful in vascular conditions accompanying diabetes due to reduced beta-cell activity (as in type 1 diabetes and in end-phase type 2 diabetes), as such patients show reduced C-peptide and insulin levels and therewith generally suffer from excess (micro) vascular permeability and excess leucocyte extravasation, together with excess circulating blood glucose.

The disclosure also provides a method for treatment of an acute and/or systemic condition of a subject suffering or believed to be suffering from the condition the method comprising providing the subject, preferably parenterally, intravenously or intraperitoneally with a chemoattractant motif W comprising autophagy-inhibiting peptide according to the disclosure, preferably a synthetic chemoattractant motif W comprising autophagy-inhibiting peptide, of at most 30 amino acids, the chemoattractant motif W comprising autophagy-inhibiting peptide provided with at least one motif PGP allowing targeting of the peptide to the CXC receptor, wherein at least one amino acid at position x is selected from the group of alanine, leucine, valine or isoleucine, the peptide also provided with at least one glutamine. It is preferred that the chemoattractant motif W comprising autophagy-inhibiting peptide comprises at least one amino acid sequence selected from the group of AQ, LQ, GQ, VQ, IQ, CQ, AQG, LQG, AQGV (SEQ ID NO:11) and LQGV (SEQ ID NO:10).

The disclosure also provides a method for treatment of a vascular and/or inflammatory condition of a human subject suffering or believed to be suffering from the condition the method comprising providing the subject, preferably parenterally, intravenously or intraperitoneally, with a hepta-, octa-, nona-, deca-, undeca- or dodeca-peptide, most preferably a hepta-, octa-, nona-peptide, provided with at least one motif PGP allowing targeting of the peptide to the CXC receptor, wherein at least one amino acid at position selected from the group of alanine, leucine, valine or isoleucine, the peptide also provided with at least one glutamine, according to the disclosure. A method is preferred wherein the peptide according to the disclosure is provided with at least two glutamines, more preferably three glutamines.

The disclosure also provides a method for treatment of an inflammatory condition of a subject suffering or believed to be suffering from the condition the method comprising providing the subject, preferably parenterally, intravenously or intraperitoneally, with a peptide having at least one amino acid sequence with LQGV (SEQ ID NO:10) and/or AQGV (SEQ ID NO:11) according to the disclosure.

The disclosure also provides a method for treatment of a vascular and/or inflammatory condition of a subject suffering or believed to be suffering from the condition the method comprising providing the subject, preferably parenterally, intravenously or intraperitoneally, with a peptide having at least one amino acid sequence with LQGV (SEQ ID NO:10) and/or AQGV (SEQ ID NO:11) according to the disclosure, preferably a synthetic peptide selected from the group AQGVAPGQ (SEQ ID NO:21), LQGVAPGQ (SEQ ID NO:22), AQGVLPGQ (SEQ ID NO:23) and LQGVLPGQ (SEQ ID NO:24).

The disclosure also provides a method for treatment of a vascular and/or inflammatory condition of a subject suffering or believed to be suffering from the condition, the method comprising providing the subject, preferably parenterally, intravenously or intraperitoneally, with a peptide having at least one amino acid sequence with LQGV (SEQ ID NO:10) and/or AQGV (SEQ ID NO:11) according to the disclosure, preferably a synthetic peptide selected from the group AQGQAPGQ (SEQ ID NO:25), LQGQAPGQ (SEQ ID NO:26), AQGQLPGQ (SEQ ID NO:27) and LQGQLPGQ (SEQ ID NO:28).

TABLE 1
Exemplary agonists of FPRs (Handbook of
biologically active Peptides, 2013: 671-680).

| Agonists | Origin |
|---|---|
| Peptides | |
| Bacteria-derived | |
| N-formyl peptides | |
| N-formy1-MLF | E. coli |
| N-formyl-MIFL | S. aureus |
| (SEQ ID NO: 29) | |
| N-formyl-MIVIL | L. monocytogenes |
| (SEQ ID NO: 30) | |
| N-formyl-MIGWI | L. monocytogenes |
| (SEQ ID NO: 31) | |
| N-formyl-MIVTLF | L. monocytogenes |
| (SEQ ID NO: 32) | |
| N-formyl-MIGWII | L. monocytogenes |
| (SEQ ID NO: 33) | |
| N-formyl-MFEDAVAWF | M. avium |
| (SEQ ID | |
| NO: 34) | |
| Mitochondria-derived | |
| N-formyl peptides | |
| N-formyl-MMYALF | Mitochondria, ND6 |
| (SEQ ID NO: 35) | |
| N-formyl-MLKLIV | Mitochondria, ND4 |
| (SEQ ID NO: 36) | |
| N-formyl-MYFINILTL | Mitochondria, ND1 |
| (SEQ ID | |
| NO: 37) | |
| N-formyl-MFADRW | Cytochrome c |
| (SEQ ID NO: 38) | oxidase subunit |
| N-formyl-Nle-LF- | Synthetic |
| Nle-YK (SEQ ID | |
| NO: 39) | |
| Mitocryptide-2 | Mitochondria |
| (MCT-2) | cytochrome b |
| Microbe-derived | |
| peptides | |
| Hp (2-20) | H. pylori |
| T20 (DP178) | HIV-1 gp41 aa. 643-678 |
| T21 (DP107) | HIV-1 gp41 aa. 558-595 |
| V3 peptide | HIV-1 gp120, V3 loop |
| N36 peptide | HIV-1 gp41 aa. 546-581 |
| F peptide | HIV-1 gp120 aa. 414-434 |
| gG-2p20 | Herpes simplex |
| | virus type 2 |
| N-formyl HKU-1 | Respiratory |
| coronavirus peptide | syndrome |
| | coronavirus |
| Host-derived peptides | |
| CKα8-1 (human CCL23) | Chemokine |
| SHAAGtide | CCL23 N-terminal 18 aa. |
| Humanin (HN) | Neuroprotective peptide |
| F2L | Heme-binding protein |
| SAA | Acute-phase protein |
| Annexin 1/ | Glucocorticoid- |
| lipocortin 1 | regulated protein |
| Ac1-25 | Annexin 1 |
| Ac2-26 | Annexin 1 |
| Ac9-25 | Annexin 1 |
| Antiflammin-2 (AF2) | Annexin 1 |
| Aα (1-42) | Amyloid precursor |
| D2D3 | uPAR (88-274) |
| SRSRYp | D2D3 |
| (SEQ ID NO: 40) | |
| LL-37 | Cathelicidin |
| PrP (106-126) | Prion protein |
| Temporin A | Rana temporaria |
| PACAP27 | Pituitary adenylate |
| | cyclase activating |
| | polypeptide |
| Agonists from peptide | |
| library | |
| WKYMVm (SEQ ID NO: 6) | Peptide library |
| WKYMVM (SEQ ID NO: 41) | Peptide library |
| MMK-1 | Peptide library |
| MMWLL (SEQ ID NO: 42), | Peptide library |
| formyl- | |
| MMWLL (SEQ ID NO: 43) | |
| CGEN-855A | Peptide library |
| Nonpeptides | |
| Host-derived | |
| nonpeptides | |
| Lipoxin A4 and | Eicosanoids |
| aspirin-triggered | |
| lipoxins | |
| Agonists from | |
| nonpeptide library | |
| Quinazolinone | Combinatorial library |
| derivative (Quin-C1) | |
| Pyrazolone, | Combinatorial library |
| 4-iodo-substituted, | |
| no. 43 | |
| AG-14 | Drug-like molecule |
| | library |
| Compound 1 and 2 | Arylcarboxylic acid |
| | hydrazide derivatives |
| Others | |
| PD168368 | Gastrin-releasing |
| PD176252 | peptide/neuromedin B |
| | receptors (BB1/BB2) |
| Trp-and Phe- | PD168368/ PD176252 |
| based analogs | |
| Related nonpeptide/ | PD168368/ PD176252 |
| nonpeptoid analogs | |
| A-71623 | Cholecystokinin-1 |
| | receptor agonist |

### BRIEF DESCRIPTION OF THE DRAWINGS

FIG. 1: Neutrophil-mediated repair response. Three possible strategies that are adopted by neutrophils to promote tissue repair. I. Neutrophils can clear necrotic cellular debris. A detailed mechanism in this progress remains to be studied, it is thought to include phagocytosis. II. Neutrophils release effectors that promote angiogenesis and regeneration; only “beneficial” effectors are listed in the figure. III. Phagocytosis of apoptotic neutrophils results in release of anti-inflammatory and reparative cytokines.

FIG. 2: Broadly, autophagy is important for immature neutrophil differentiation and mature function. Several studies suggest a dual role for autophagy in neutrophil function during inflammation. Augmentation of autophagy may be an effective target for enhancement of proper myeloid differentiation and antimicrobial defense, inducing increased NET formation, degranulation and inflammatory cytokine release. Conversely, autophagy inhibition may be useful in neutrophil-mediated inflammatory disease (Korean J. Physiol. Pharmacol. 2020 Jan.; 24(1): 1-10). Inhibition of autophagy reverses autophagic neutrophil death and slows disease development [Oncotarget. 2017 Sep. 26; 8(43):74720-74735]. Inhibition of autophagy during neutrophil-mediated inflammation and autoimmune disease reduced disease severity and progression by suppressing degranulation and ROS production [PLoS One. 2012; 7(12):e51727, ncbi.nlm.nih.gov/pmc/articles/PMC6940497/—B35 Nature. 2015 Dec. 24; 528(7583):565-9]. Similarly, suppression of autophagy through NLRP3 knockdown or inhibition of the NLRP3 inflammasome enhanced neutrophil recruitment and phagocytosis, thereby improving bacterial clearance and augmenting the survival of septic mice [J. Immunol. 2017 Feb. 1; 198(3):1253-1262]. Depending on the inflammatory environment, autophagy in neutrophil may behave as a double-edged sword. While it is beneficial in combating infection, it may favor excessive inflammation through exaggerated NETs formation and cytokine release. Thus, autophagic homeostasis is important for proper neutrophil effector function and host health.

FIG. 3: FPRs are a family of three human receptors (FPR1, FPR2, and FPR3). FPR1 was first identified to bind bacterial formyl-methionyl-leucyl-phenylalanine (fMLF). FPRs are essential for host defense against the invasion of pathogens, malignancies, and expansion of traumas, whereas abnormal expression of FPR function can be harmful. FPRs are also subject to homologous and heterologous desensitization (of other chemoattractant receptors): excessive activation of the receptor by a ligand causes the unresponsiveness of the receptors to subsequent stimulation by the same or other ligands. Therefore, desensitization of immune-competent cells could be detrimental for host defense. Human mitochondrial formylated peptides derived from cell death activate FPR1 signaling, and are recognized as key drivers of ALI/ARDS [Thorax. 2017; 72:928-936]. FPR1 inhibitors (such as cyclosporin H) preserve normal neutrophil bacterial phagocytosis or superoxide production in response to infections. Therefore, mitigating FPR1 homologous and heterologous desensitization can protect the host from systemic sterile inflammation and secondary infection following tissue injury or primary infection. Formyl-peptide-receptor mediated-vascular permeability occurs after cell and tissue trauma. Mitochondrial N-formyl peptides (F-MIT) released from trauma/cell damage activate formyl peptide receptor (FPR) leading to changes in endothelial cell cytoskeleton, which subsequently induces endothelial contraction and vascular permeability, leukocyte extravasation and hypotension. N-Formyl peptides are common molecular signatures of bacteria and mitochondria that activate the formyl peptide receptor (FPR). FPR activation by mitochondrial N-formyl peptides (F-MIT) elicits changes in cytoskeleton-regulating proteins in endothelial cells that lead to increased endothelial cell contractility with increased vascular leakage and extravasation of leukocytes. FPR activation via mitochondrial N-formyl peptides (F-MIT) originating from tissue damage after injury such as trauma is a key contributor to impaired barrier function following cell and tissue injury or trauma, resulting in detrimental vascular effects such as adverse vascular permeability with edema, vascular leakage, adverse leukocyte extravasation and hypotension.

FIG. 4: Intracellular trafficking of activated receptors. Agonist dependent phosphorylation of the receptors leads to the recruitment of β-arrestins. The receptor-β-arrestin complex is targeted to clathrin-coated pits, traffics in early endosomes and accumulates in a perinuclear recycling compartment. After dephosphorylation and dissociation from β-arrestins, the receptors resensitize and recycle to the cell surface. In the case of C5aR, a fraction of the internalized receptor is targeted to lysosomes for degradation.

FIG. 5: Graphic description of p38-MK2-HSP27 pathway (left) and PI3K/AKT/mTOR pathway (right) involved in regulation of endothelial cell-cytoskeleton organization.

FIG. 6: Formyl-peptide-receptor-mediated peptide effects at 20 ng/ml (left-hand panels) or 50 ng/ml (right-hand panels. FPR-activation of FPR-expressing cells with prototype FPR-ligand fMLP causes rapidly induced and significant (p<0.05; p38 from 60 to 600 seconds, PKB at 600 seconds) changes in phosphorylation status of PKB (also known as AKT) (FIG. 6A) and p38 MAPK kinases (FIG. 6C), but not (or not detectable) in STAT3, JNK (FIG. 6B) and P42/p44MAPK/ERK1,2 (FIG. 6D) kinases. AQGV (SEQ ID NO:11) peptide effects on p38 MAPK (FIG. 6C) are already detected at 30 seconds after FPR-stimulation, AQGV (SEQ ID NO:11) peptide effects on PKB(AKT) follow (FIG. 6A) in a bi-phasic pattern at 300 seconds. Both AQGV (SEQ ID NO:11) peptide effects on p38 and PKB-mediated signalling last for the full 600 seconds tested whereas the other kinases tested were not affected throughout. This acute and specific response to treatment shows specific and rapid effects of autophagy-inhibiting peptide on p38 signaling in the context of regulation of the PI3K/AKT/mTOR pathway. The pathway is governing the balance between proteolysis and proteogenesis regulating cytoskeleton changes affecting vascular permeability. It is shown that an autophagy-inhibiting peptide reduces p38 MAPK kinase activated changes as well as reduces PI3K/AKT/mTOR activated induced changes in cell cytoskeleton reorganization affecting endothelial cell contraction and adverse vascular permeability. Autophagy-inhibiting peptide is useful and capable of addressing adverse vascular permeability, such as manifested by edema with vascular leakage, adverse leukocyte extravasation and hypotension, tissue injury and immune responses in human subjects.

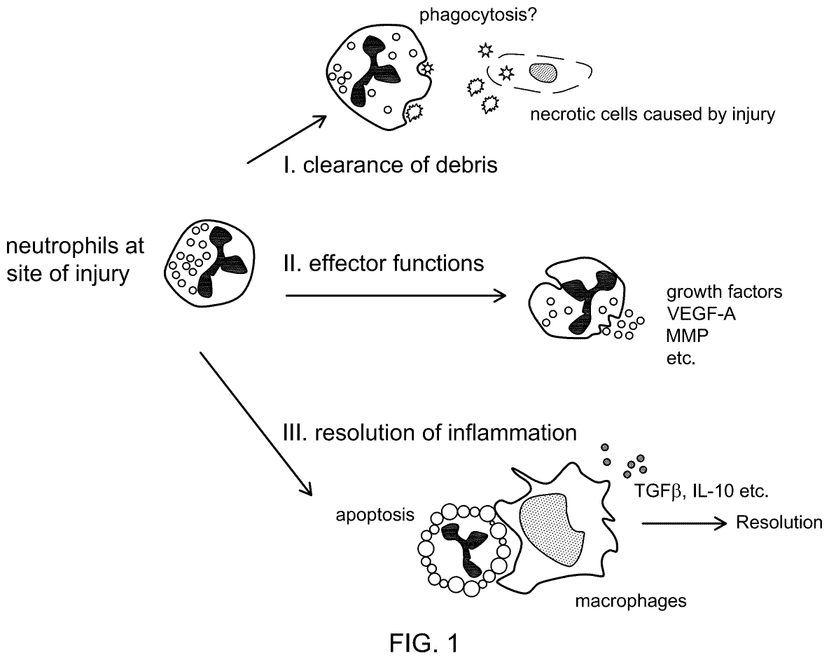

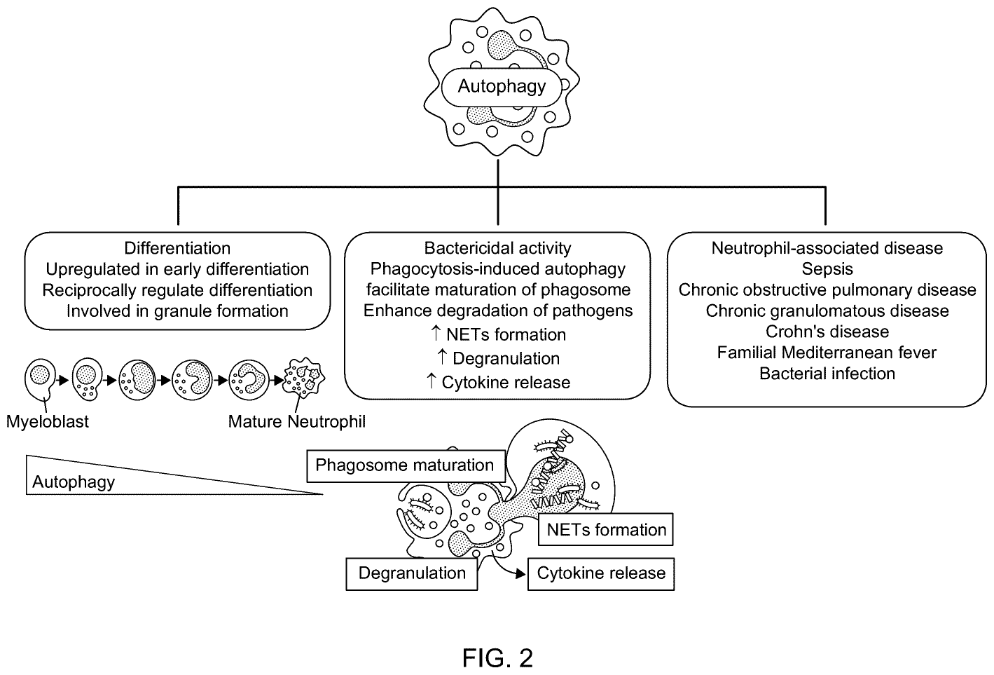

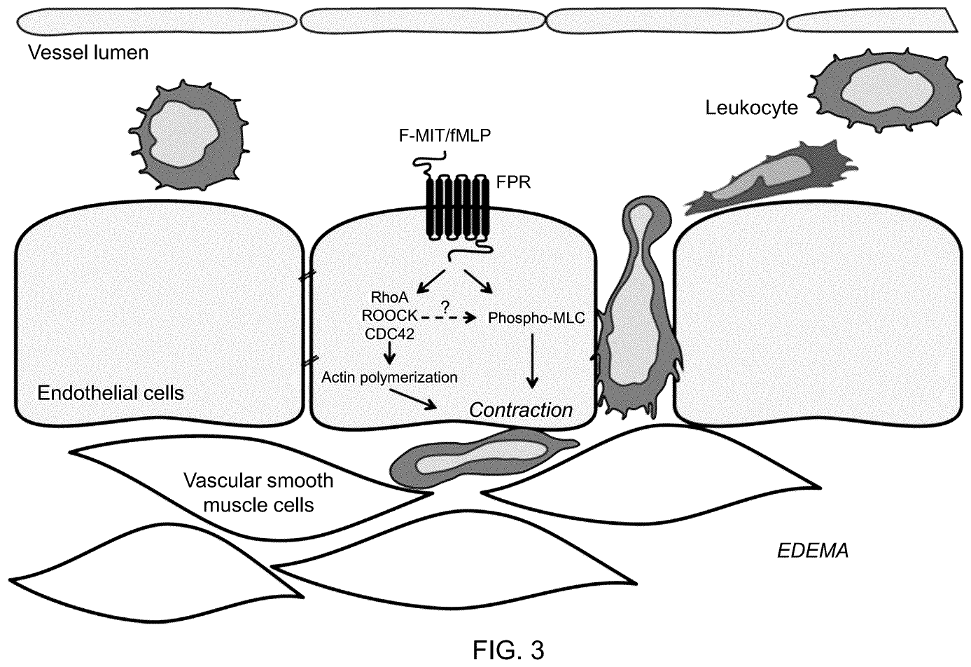

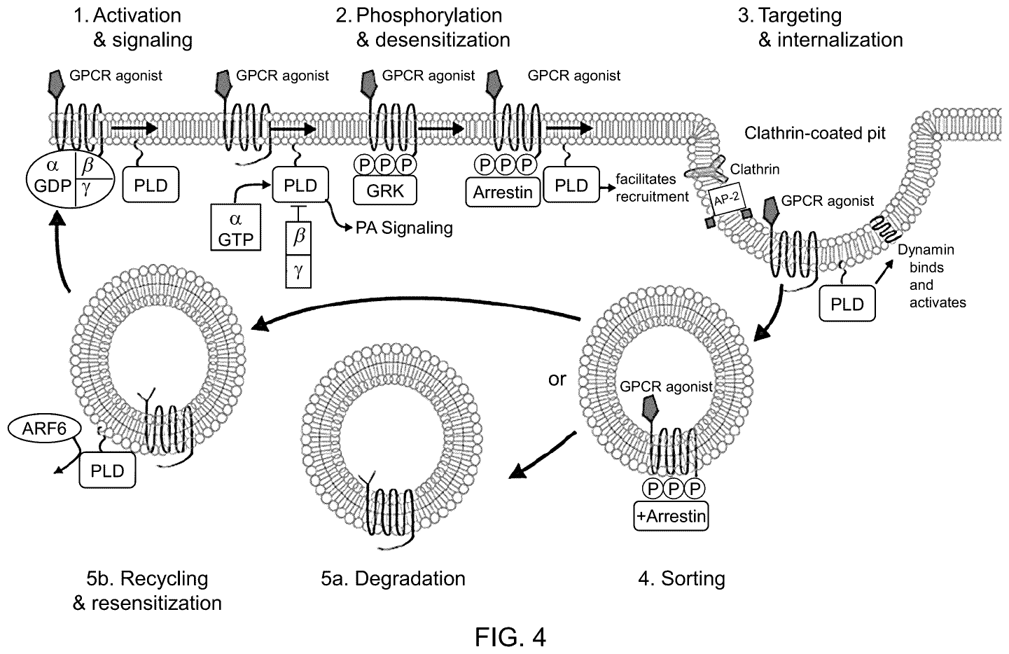

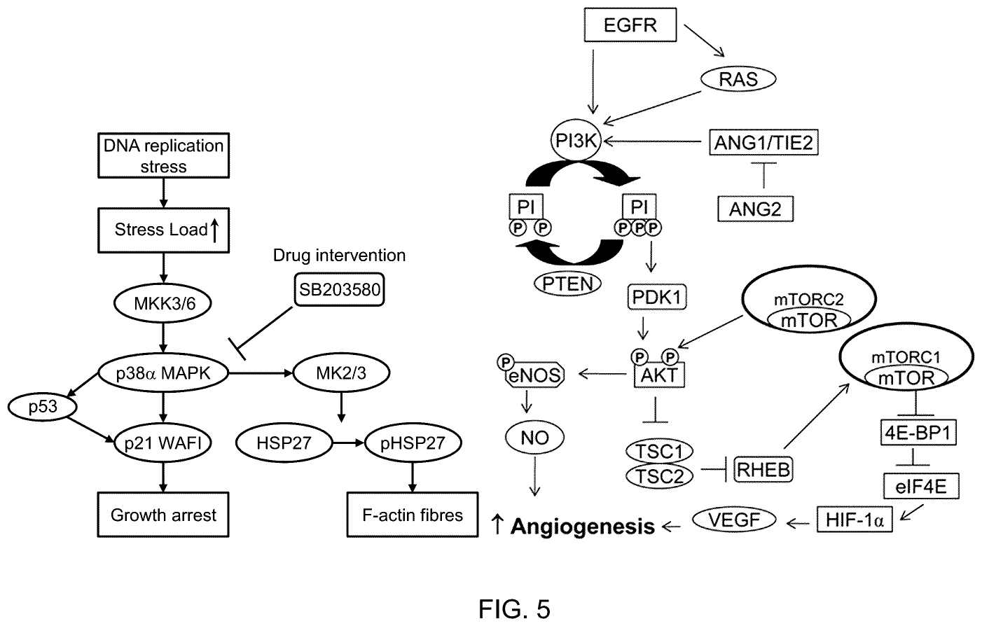

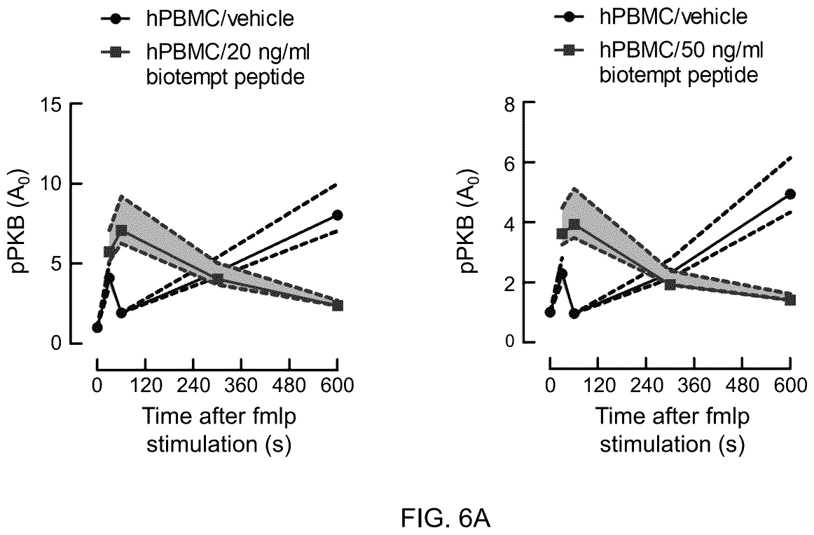

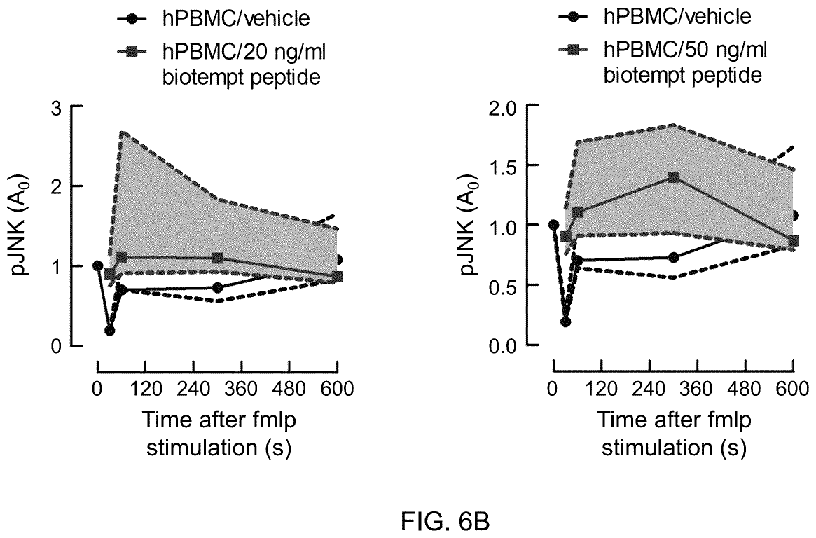

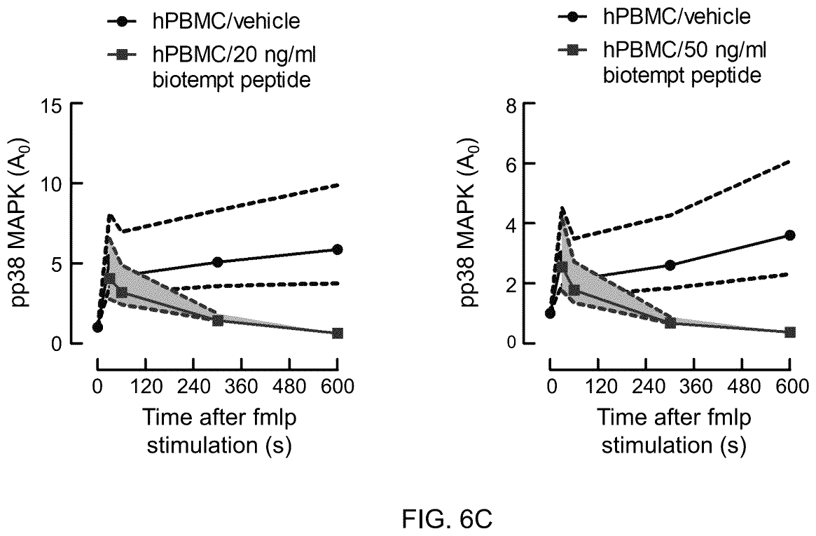

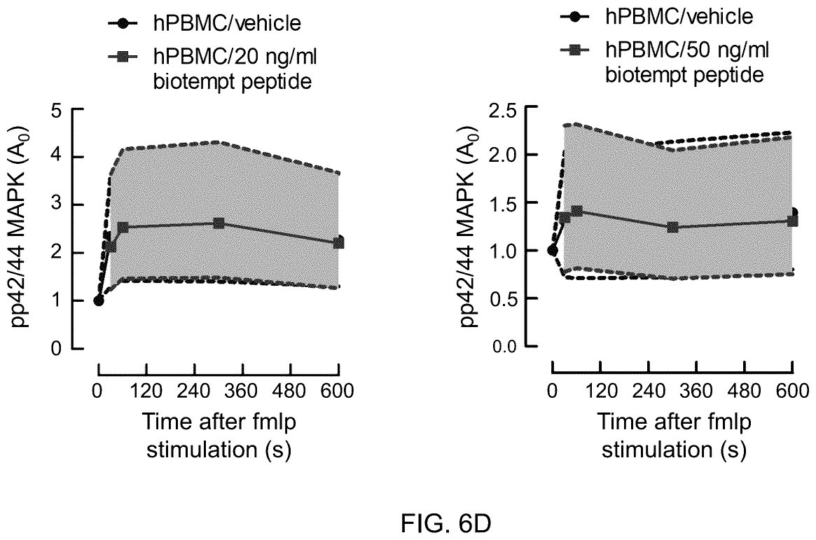

### DETAILED DESCRIPTION

In another embodiment, a chemoattractant motif W comprising autophagy-inhibiting peptide, or a functional analogue thereof, is provided for use in the treatment of a human subject having impaired kidney function. In a further embodiment, the impaired kidney function is acute kidney injury (AKI). In one embodiment, a chemoattractant motif W comprising autophagy-inhibiting peptide, or a functional analogue thereof, is provided for use in the treatment of a human subject for improving kidney function. Kidney function can be assessed by determining the glomerular filtration rate (GFR), for example, by assessing the clearance of iohexol from blood plasma. Kidney function can also be assessed by measuring plasma levels of creatinine and calculating an estimated GFR (eGFR) function therefrom, also referred to as the MDRD formula or equation, taking into account patient characteristics such as sex, age and race (Modification of Diet in Renal Disease). Kidney function can be assessed based on GFR measurements (or estimates thereof based on MDRD) by applying the RIFLE criteria. Having a RIFLE score that is in the stage of risk, injury, failure, loss or ESKD, can be indicative of kidney injury and/or impairment of kidney function. Assessing kidney function in humans is standard clinical practice (e.g., by determining GFR, creatinine clearance, and/or eGFR/MDRD). Improvements in kidney function as compared with not receiving the chemoattractant motif W comprising autophagy-inhibiting peptide can include progressing to a kidney function stage as assessed under the RIFLE criteria to a less severe stage (e.g., a patient progressing from having injury to being at risk of injury or having no AKI). Improvements in kidney function also include having an improvement in GFR or eGFR scores. Irrespective of what assessment is made, the use of the chemoattractant motif W comprising autophagy-inhibiting peptide, or analogue thereof, can improve kidney function in humans having kidney injury and/or an impairment of kidney function in subjects absent of immunomodulatory effects.

Not only does the use of the chemoattractant motif W comprising autophagy-inhibiting peptide allow for improving kidney function it can also prevent a reduction and/or an impairment of kidney function. Accordingly, AKI may be prevented. Preferably, in prevention of a human subject having impaired kidney function, the chemoattractant motif W comprising autophagy-inhibiting peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the chemoattractant motif W comprising autophagy-inhibiting peptide is administered for at least 1 hour, more preferably at least 1.5 hours, most preferably at least 2 hours. Preferably, the administration of the chemoattractant motif W comprising autophagy-inhibiting peptide is at a rate of at least 10 mg/kg/hr and administered for at least 1 hour, more preferably at least 1.5 hours, most preferably at least 2 hours, such as at least 2.5 hours. Hence, in one embodiment, the use of the chemoattractant motif W comprising autophagy-inhibiting peptide, or analogue thereof, allows to maintain kidney function in human patients. In another embodiment, the use of the chemoattractant motif W comprising autophagy-inhibiting peptide, or analogue thereof, allows to prevent a reduction and/or impairment of kidney function in human patients. For example, a human patient that may be classified as having no AKI, or being at risk of having kidney injury (such as AKI), when such a patient receives treatment with the chemoattractant motif W comprising autophagy-inhibiting peptide, such a patient may maintain its status instead of progressing to a kidney function that is a more severe stage. Hence, human patients that are at risk of developing kidney injury, e.g., due to (induced) trauma, such human patients as a result of receiving treatment with the chemoattractant motif W comprising autophagy-inhibiting peptide, or analogue thereof, can maintain their kidney function status.

In another embodiment, the use of a chemoattractant motif W comprising autophagy-inhibiting peptide, or a functional analogue thereof, reduces adverse fluid retention in the human subject. Fluid retention or fluid overload can occur in human subjects, symptoms of which, e.g., include weight gain and edema. Fluid retention can be the result of reduced kidney function and/or diabetes type 1 or end-phase type 2 (when no or little endogenous C-peptide is produced by the subject and micro-vascular flow is compromised). Fluid retention can be the result of leaky capillaries. Hence, the use of chemoattractant motif W comprising autophagy-inhibiting peptide, and analogues thereof, may have an effect on the leakiness of capillaries, reducing leakage of plasma and extravasation of immune cells (leucocytes) from the blood to peripheral tissue and/or organs. Most preferably edema and/or leukocyte extravasation is reduced and/or avoided by the use of chemoattractant motif W comprising autophagy-inhibiting peptide. Such may also be referred to as adverse fluid retention as it has an adverse effect on the patient. Whichever is the cause of fluid retention, the use of a chemoattractant motif W comprising autophagy-inhibiting peptide, or a functional analogue thereof, can improve fluid retention (with or without extravasated leucocytes) in human subjects thereby alleviating symptoms associated with fluid retention such as weight gain and edema, which subsequently can reduce the use of diuretics. Preferably, in use of (in particular, the smaller) chemoattractant motif W comprising autophagy-inhibiting peptides (AIPs of preferably 5-15 amino acids long) to improve fluid retention, the chemoattractant motif W comprising autophagy-inhibiting peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the chemoattractant motif W comprising autophagy-inhibiting peptide is administered for at least 1 hour, more preferably at least 1.5 hours, most preferably at least 2 hours. Preferably, the administration of the chemoattractant motif W comprising autophagy-inhibiting peptide is at a rate of at least 20 mg/kg/hr and administered for at least 1 hour, more preferably at least 1.5 hours, most preferably at least 2 hours, such as at least 2.5 hours.

In another embodiment, the use of the chemoattractant motif W comprising autophagy-inhibiting peptide, or a functional analogue thereof, in accordance with the disclosure, is not restricted to patients having kidney injury, neither to patients having beta-cell failure. The use of a chemoattractant motif W comprising autophagy-inhibiting peptide, or a functional analogue thereof, in accordance with the disclosure, includes the treatment of human patients that are believed to be at risk of having a systemic inflammation and/or are anticipated to require anti-inflammatory therapy. Such human patients include patients that are to be admitted, or are expected to be admitted, into intensive care. Hence, the use of the chemoattractant motif W comprising autophagy-inhibiting peptide, or a functional analogue thereof, includes a use for induced trauma, such as surgery. Induced trauma includes any physical injury to the human body and typically can include the loss of blood and/or injury to tissues of the human subject. Induced trauma includes, e.g., surgery. Hence, in a preferred embodiment, the induced trauma is surgery. The use of the chemoattractant motif W comprising autophagy-inhibiting peptide for treatment of induced trauma, such as surgery, may be before, during and/or after surgery. It may be preferred that the use of the chemoattractant motif W comprising autophagy-inhibiting peptide, or an analogue thereof, is during surgery. Preferably, the chemoattractant motif W comprising autophagy-inhibiting peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the chemoattractant motif W comprising autophagy-inhibiting peptide is administered for at least 1 hour, more preferably at least 1.5 hours, most preferably at least 2 hours. Preferably, the administration of the chemoattractant motif W comprising autophagy-inhibiting peptide is at a rate of at least 20 mg/kg/hr and administered for at least 1 hour, more preferably at least 1.5 hours, most preferably at least 2 hours, such as at least 2.5 hours.

In another, or further, embodiment, the use of a chemoattractant motif W comprising autophagy-inhibiting peptide, or a functional analogue thereof, for use in accordance with the disclosure is for use is in a human subject having heart failure. Preferably, in use in a human subject having heart failure, the chemoattractant motif W comprising autophagy-inhibiting peptide is administered at a rate that is at least 10 mg/kg patient weight per hour (mg/kg/hr). Preferably, the administration rate is at least 20 mg, at least 30, at least 40 or, most preferably, at least 50 mg/kg/hr. Preferably, the chemoattractant motif W comprising autophagy-inhibiting peptide is administered for at least 1 hour, more preferably at least 1.5 hours, most preferably at least 2 hours. Preferably, the administration of the chemoattractant motif W comprising autophagy-inhibiting peptide is at a rate of at least 20 mg/kg/hr and administered for at least 1 hour, more preferably at least 1.5 hours, most preferably at least 2 hours, such as at least 2.5 hours.

The disclosure includes the use of a chemoattractant motif W comprising autophagy-inhibiting peptide, or a functional analogue thereof, for use in the treatment of a human subject considered at risk or suffering from fluid overload, the use comprising modifying fluid retention in the human subject. The use of chemoattractant motif W comprising autophagy-inhibiting peptide, or a functional analogue thereof, in accordance with the disclosure, includes the treatment of human patients that are believed to be at risk of having fluid overload and/or anticipated to require hemodynamic therapy. Such human patients include patients that are to be admitted, or are expected to be admitted, into intensive care. Hence, the use of chemoattractant motif W comprising autophagy-inhibiting peptide, or a functional analogue thereof, includes a use for prevention of induced fluid overload, such as with fluid therapy. Preferably, in use for prevention of induced fluid overload, the chemoattractant motif W comprising autophagy-inhibiting peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the chemoattractant motif W comprising autophagy-inhibiting peptide is administered for at least 1 hour, more preferably at least 1.5 hours, most preferably at least 2 hours. Preferably, the administration of the chemoattractant motif W comprising autophagy-inhibiting peptide is at a rate of at least 20 mg/kg/hr and administered for at least 1 hour, more preferably at least 1.5 hours, most preferably at least 2 hours, such as at least 2.5 hours. In another embodiment, the use of chemoattractant motif W comprising autophagy-inhibiting peptide, or a functional analogue thereof, in accordance with the disclosure, is not restricted to patients having kidney injury and/or requiring hemodynamic therapy.

The disclosure includes the use of a chemoattractant motif W comprising autophagy-inhibiting peptide, or a functional analogue thereof, for use in the treatment of a human subject to improve the subject's length of stay at the ICU, further to shorten the subject's length of stay at the ICU. One way in which this may be attained is by modifying fluid retention in the human subject. The use of chemoattractant motif W comprising autophagy-inhibiting peptide, or a functional analogue thereof, in accordance with the disclosure, includes the treatment of human patients that are believed to be at risk from treatment with a vasopressor or an inotropic medication and/or anticipated to require hemodynamic therapy with fluid therapy. Such human patients include patients that are or are to be admitted, or are expected to be admitted, into intensive care, and for which shortening length-of-stay at ICU is desired. Hence, the use of chemoattractant motif W comprising autophagy-inhibiting peptide, or a functional analogue thereof, includes a use for the treatment of human patients that are believed to be at risk from treatment with detrimental vasopressor or inotropic medication and/or with fluid therapy, is provided as shown, e.g., in the examples. Preferably, in use for shortening a subject's length of stay at the ICU, in human patients that are believed to be at risk, the chemoattractant motif W comprising autophagy-inhibiting peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the chemoattractant motif W comprising autophagy-inhibiting peptide is administered for at least 1 hour, more preferably at least 1.5 hours, most preferably at least 2 hours. Preferably, the administration of the chemoattractant motif W comprising autophagy-inhibiting peptide is at a rate of at least 20 mg/kg/hr and administered for at least 1 hour, more preferably at least 1.5 hours, most preferably at least 2 hours, such as at least 2.5 hours.

In another embodiment, the use of chemoattractant motif W comprising autophagy-inhibiting peptide, or a functional analogue thereof, in accordance with the disclosure, is not restricted to patients having kidney injury, beta-cell failure and/or requiring hemodynamic therapy. The disclosure includes the use of a chemoattractant motif W comprising autophagy-inhibiting peptide, or a functional analogue thereof, for use in the treatment of a human subject to improve the subject's length of stay at the hospital, further to shorten the subject's length of stay at the hospital, the use comprising modifying fluid retention in the human subject. The use of chemoattractant motif W comprising autophagy-inhibiting peptide, or a functional analogue thereof, in accordance with the disclosure, includes the treatment of human patients that are believed to be at risk from treatment with a vasopressor or an inotropic medication and/or anticipated to require hemodynamic therapy with fluid therapy. Such human patients include patients that are or are to be admitted, or are expected to be admitted, into intensive care or hospital, and for which shortening length-of-stay at hospital is desired. Hence, the use of chemoattractant motif W comprising autophagy-inhibiting peptide, or a functional analogue thereof, includes a use for the treatment of human patients that are believed to be at risk from treatment with detrimental vasopressor or inotropic medication and/or with fluid therapy, is provided. Preferably, in use for shortening a subject's length of stay at the ICU, in human patients that are believed to be at risk, the chemoattractant motif W comprising autophagy-inhibiting peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the chemoattractant motif W comprising autophagy-inhibiting peptide is administered for at least 1 hour, more preferably at least 1.5 hours, most preferably at least 2 hours. Preferably, the administration of the chemoattractant motif W comprising autophagy-inhibiting peptide is at a rate of at least 20 mg/kg/hr and administered for at least 1 hour, more preferably at least 1.5 hours, most preferably at least 2 hours, such as at least 2.5 hours.

Preferably, the use of the chemoattractant motif W comprising autophagy-inhibiting peptide, or a functional analogue thereof, in accordance with the disclosure and as described above, involves the administration of the peptide into the bloodstream. It is understood that administration into the bloodstream comprises, e.g., intravenous administration or intra-arterial administration. A constant supply of chemoattractant motif W comprising autophagy-inhibiting peptide, or an analogue thereof, is preferred, e.g., via an infusion wherein the chemoattractant motif W comprising autophagy-inhibiting peptide, or analogue thereof, is comprised in a physiological acceptable solution. Suitable physiologically acceptable solutions may comprise physiological salt solutions (e.g., 0.9% NaCl) or any other suitable solution for injection and/or infusion. Such physiological solutions may comprise further compounds (e.g., glucose etc.) that may further benefit the human subject, and may also include other pharmaceutical compounds (e.g., vasopressors).

Preferably, the chemoattractant motif W comprising autophagy-inhibiting peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the chemoattractant motif W comprising autophagy-inhibiting peptide is administered for at least 1 hour, more preferably at least 1.5 hours, most preferably at least 2 hours. Preferably, the administration of the chemoattractant motif W comprising autophagy-inhibiting peptide is at a rate of at least 20 mg/kg/hr and administered for at least 1 hour, more preferably at least 1.5 hours, most preferably at least 2 hours, such as at least 2.5 hours. Preferably, the administration is during surgery. More preferably, the administration is during the entire duration of surgery.

In another embodiment, a chemoattractant motif W comprising autophagy-inhibiting peptide, or a functional analogue thereof, is provided for any use in accordance with the disclosure as described above, wherein the human subject is admitted to intensive care, and wherein the use improves parameters measured of the human subject, the parameters of the human subject determined to assess to remain in intensive care or not. As shown above, parameters that are assessed when a human patient is in intensive care include parameters related to kidney function and fluid retention, allowing for improved hemodynamic stability. In any case, the use of the chemoattractant motif W comprising autophagy-inhibiting peptide, or analogue thereof, is to improve such parameters to thereby reduce the length of stay in the intensive care unit. Not only does the use of the chemoattractant motif W comprising autophagy-inhibiting peptide, or analogue thereof reduce the length of stay in the intensive care, the effect of the use of the chemoattractant motif W comprising autophagy-inhibiting peptide, or analogue thereof, also reduces the length of stay in the hospital and reduces readmittance into the hospital. Further embodiments.

Further embodiment 1: A molecule with a chemoattractant motif W, preferably a neutrophil-chemoattractant motif, the molecule also comprising an autophagy-inhibiting peptide, or a functional analogue thereof, for use in the treatment of a human subject, the use comprising modifying beta-cell function in the human subject.

Further embodiment 2: A molecule with a chemoattractant motif W, preferably a neutrophil-chemoattractant motif, the molecule also comprising an autophagy-inhibiting peptide, or a functional analogue thereof, for use in the treatment of a human subject considered at risk or suffering from beta-cell failure, the use comprising modifying pre-pro-insulin levels in the human subject.

Further embodiment 3: A molecule with a chemoattractant motif W, preferably a neutrophil-chemoattractant motif, the molecule also comprising an autophagy-inhibiting peptide, or a functional analogue thereof, for use in the treatment of a human subject having impaired kidney function, the use comprising modifying fluid retention in the human subject.

Further embodiment 4: A molecule with a chemoattractant motif W, preferably a neutrophil-chemoattractant motif, the molecule also comprising an autophagy-inhibiting peptide, or a functional analogue thereof, for use in the treatment of a human subject, the use comprising modifying inflammation in the human subject.

Further embodiment 5: A molecule with a chemoattractant motif W, preferably a neutrophil-chemoattractant motif, the molecule also comprising an autophagy-inhibiting peptide, or a functional analogue thereof, for use in the treatment of a human subject, the use comprising modifying fluid retention in the human subject.

Further embodiment 6: A molecule with a chemoattractant motif W, preferably a neutrophil-chemoattractant motif, the molecule also comprising an autophagy-inhibiting peptide, or a functional analogue thereof, for use in the treatment of a human subject considered at risk or suffering from excess vasopressor/inotropic use, the use comprising modifying fluid retention in the human subject.

Further embodiment 7: A molecule with a chemoattractant motif W, preferably a neutrophil-chemoattractant motif, the molecule also comprising an autophagy-inhibiting peptide, or a functional analogue thereof, for use in the treatment of a human subject, wherein the human subject is subjected to induced trauma and wherein the use comprises modifying fluid retention in the human subject.

Further embodiment 8: A molecule with a chemoattractant motif W, preferably a neutrophil-chemoattractant motif, the molecule also comprising an autophagy-inhibiting peptide, or a functional analogue thereof, for use in the treatment of a human subject considered at risk or suffering from fluid overload, the use comprising modifying fluid retention in the human subject.

Further embodiment 9: A molecule with a chemoattractant motif W, preferably a neutrophil-chemoattractant motif, the molecule also comprising an autophagy-inhibiting peptide, or a functional analogue thereof, for use in the treatment of a human subject having impaired kidney function, the use comprising modifying fluid retention in the human subject.

Further embodiment 10: A molecule with a chemoattractant motif W, preferably a neutrophil-chemoattractant motif, the molecule also comprising an autophagy-inhibiting peptide, or a functional analogue thereof, for use as in accordance with any one of further embodiments 1-9, wherein the use reduces fluid retention in the human subject.

Further embodiment 11: A molecule with a chemoattractant motif W, preferably a neutrophil-chemoattractant motif, the molecule also comprising an autophagy-inhibiting peptide, or a functional analogue thereof, for use in accordance with any one of further embodiments 1-10, wherein the use comprises a reduced use of vasopressive agents.

Further embodiment 12: A molecule with a chemoattractant motif W, preferably a neutrophil-chemoattractant motif, the molecule also comprising an autophagy-inhibiting peptide, or a functional analogue thereof, for use in accordance with any one of further embodiments 1-11, wherein the use comprises a reduced fluid intake.

Further embodiment 13: A molecule with a chemoattractant motif W, preferably a neutrophil-chemoattractant motif, the molecule also comprising an autophagy-inhibiting peptide, or a functional analogue thereof, for use in accordance with further embodiment 7, wherein the reduced use of vasopressive agents comprises a reduced duration of vasopressive agent use.

Further embodiment 14: A molecule with a chemoattractant motif W, preferably a neutrophil-chemoattractant motif, the molecule also comprising an autophagy-inhibiting peptide, or a functional analogue thereof, for use in accordance with any one of further embodiments 6-9, wherein the subject is subjected to induced trauma.

Further embodiment 15: A molecule with a chemoattractant motif W, preferably a neutrophil-chemoattractant motif, the molecule also comprising an autophagy-inhibiting peptide, or a functional analogue thereof, for use in accordance with any one of further embodiments 8-12, wherein the use improves kidney function in the human subject.

Further embodiment 16: A molecule with a chemoattractant motif W, preferably a neutrophil-chemoattractant motif, the molecule also comprising an autophagy-inhibiting peptide, or a functional analogue thereof, for use in accordance with further embodiment 11, wherein the improved kidney function involves an improved GFR rate.

Further embodiment 17: A molecule with a chemoattractant motif W, preferably a neutrophil-chemoattractant motif, the molecule also comprising an autophagy-inhibiting peptide, or a functional analogue thereof, for use in accordance with any one of further embodiments 6-12, wherein the human subject has impaired kidney function the impaired kidney function being AKI.

Further embodiment 18: A molecule with a chemoattractant motif W, preferably a neutrophil-chemoattractant motif, the molecule also comprising an autophagy-inhibiting peptide, or a functional analogue thereof, for use as in accordance with any one of further embodiments 1-13, wherein the use reduces leakage of plasma and extravasation of blood from the blood to peripheral tissue and/or organs.

Further embodiment 19: A molecule with a chemoattractant motif W, preferably a neutrophil-chemoattractant motif, the molecule also comprising an autophagy-inhibiting peptide, or a functional analogue thereof, for use as in accordance with any one of further embodiments 1-13, wherein the use reduces leakage of plasma from the blood to peripheral tissue and/or organs.

Further embodiment 20: A molecule with a chemoattractant motif W, preferably a neutrophil-chemoattractant motif, the molecule also comprising an autophagy-inhibiting peptide, or a functional analogue thereof, for use as in accordance with any one of further embodiments 1-13, wherein the use reduces extravasation of blood from the blood to peripheral tissue and/or organs.

Further embodiment 21: A molecule with a chemoattractant motif W, preferably a neutrophil-chemoattractant motif, the molecule also comprising an autophagy-inhibiting peptide, or a functional analogue thereof, for use in accordance with any one of further embodiments 1-20, wherein the use is in a human subject suffering from or at risk of heart failure.

Further embodiment 22: A molecule with a chemoattractant motif W, preferably a neutrophil-chemoattractant motif, the molecule also comprising an autophagy-inhibiting peptide, or a functional analogue thereof, for use in accordance with any one of further embodiments 1-21, wherein the use is in a human subject at risk of having edema.

Further embodiment 23: A molecule with a chemoattractant motif W, preferably a neutrophil-chemoattractant motif, the molecule also comprising an autophagy-inhibiting peptide, or a functional analogue thereof, for use in accordance with any one of further embodiments 7 and 14, wherein the human subject has been subjected to induced trauma, the induced trauma being surgery.

Further embodiment 24: A molecule with a chemoattractant motif W, preferably a neutrophil-chemoattractant motif, the molecule also comprising an autophagy-inhibiting peptide, or a functional analogue thereof, for use in accordance with further embodiment 23, wherein the surgery requires a cardiopulmonary bypass.

Further embodiment 25: A molecule with a chemoattractant motif W, preferably a neutrophil-chemoattractant motif, the molecule also comprising an autophagy-inhibiting peptide, or a functional analogue thereof, for use in accordance with any one of further embodiments 1-24, wherein the peptide is administered into the bloodstream.

Further embodiment 26: A molecule with a chemoattractant motif W, preferably a neutrophil-chemoattractant motif, the molecule also comprising an autophagy-inhibiting peptide, or a functional analogue thereof, for use in accordance with further embodiment 25, wherein the peptide is administered at a rate of at least 10 mg/kg body weight/hour.

Further embodiment 27: A molecule with a chemoattractant motif W, preferably a neutrophil-chemoattractant motif, the molecule also comprising an autophagy-inhibiting peptide, a functional analogue thereof, for use in accordance with further embodiment 25 or further embodiment 26, wherein the peptide is administered for at least 1 hour.

Further embodiment 28: A molecule with a chemoattractant motif W, preferably a neutrophil-chemoattractant motif, the molecule also comprising an autophagy-inhibiting peptide, or a functional analogue thereof, for use in accordance with any one of further embodiments 1-27, wherein the human subject is admitted into intensive care, and wherein the use improves parameters measured of the human subject, the parameters of the human subject determined to assess remaining in intensive care.

Further embodiment 29: A molecule with a chemoattractant motif W, preferably a neutrophil-chemoattractant motif, the molecule also comprising an autophagy-inhibiting peptide, or a functional analogue thereof, for use in accordance with further embodiment 27, wherein the improvement in parameters results in a reduced length of stay at intensive care.

Further embodiment 30: A molecule with a chemoattractant motif l, preferably a neutrophil-chemoattractant motif, the molecule also comprising an autophagy-inhibiting peptide, or a functional analogue thereof, for use as in accordance with any one of further embodiments 1-29, wherein the use induces vasoconstriction.

Further embodiment 31: A method of treatment comprising administering a molecule with a chemoattractant motif W, preferably a neutrophil-chemoattractant motif, the molecule also comprising an autophagy-inhibiting peptide, or a functional analogue thereof, to a human subject, the human subject being in need of maintaining hemodynamic stability.

Further embodiment 32: A method of treatment comprising administering a molecule with a chemoattractant motif W, preferably a neutrophil-chemoattractant motif, the molecule also comprising an autophagy-inhibiting peptide, or a functional analogue thereof, to a human subject, the human subject being in need of improving hemodynamic stability.

Further embodiment 33: A method of treatment comprising administering a molecule with a chemoattractant motif W, preferably a neutrophil-chemoattractant motif, the molecule also comprising an autophagy-inhibiting peptide, or a functional analogue thereof, to a human subject, the human subject having impaired kidney function, wherein the treatment of administering a chemoattractant motif W comprising autophagy-inhibiting peptide comprises maintaining or improving hemodynamic stability in the human subject.

Further embodiment 34: A molecule with a chemoattractant motif W, preferably a neutrophil-chemoattractant motif, the molecule also comprising an autophagy-inhibiting peptide, comprising a synthetic peptide or functional analogue thereof, provided with a glutamine (Q) and a CXC-receptor(ER) binding amino acid sequence motif and also comprising at least 50% amino acids selected from the group of autophagy-inhibiting amino acids alanine (in one letter code: A), glutamine (Q), glycine (G), valine (V), leucine (L), proline (P), and arginine (R).

Further embodiment 35: A molecule with a chemoattractant motif W, preferably a neutrophil-chemoattractant motif, the molecule also comprising an autophagy-inhibiting peptide, or functional analogue thereof, according to further embodiment 34, comprising at least one amino acid sequence selected from the group of AQ, LQ, GQ, VQ, AQG, LQG, AQGV (SEQ ID NO:11) and LQGV (SEQ ID NO:10).

Further embodiment 36: A molecule with a chemoattractant motif W, preferably a neutrophil-chemoattractant motif, the molecule also comprising an autophagy-inhibiting peptide, consisting of a hepta-, octa-, nona-, deca-, undeca- or dodeca-peptide according to further embodiment 34 or 35.

Further embodiment 37: A molecule with a chemoattractant motif W, preferably a neutrophil-chemoattractant motif, the molecule also comprising an autophagy-inhibiting peptide, according to any one of further embodiments 34 to 36 provided with at least two glutamines.

Further embodiment 38: A pharmaceutical composition comprising A molecule with a chemoattractant motif W, preferably a neutrophil-chemoattractant motif, the molecule also comprising an autophagy-inhibiting peptide, according to any one of further embodiments 34 to 40.

Further embodiment 39: A pharmaceutical composition according to further embodiment 38 additionally comprising an insulin.

Further embodiment 40: A molecule with a chemoattractant motif W, preferably a neutrophil-chemoattractant motif, the molecule also comprising an autophagy-inhibiting peptide, according to any one of further embodiments 3 to 37, or a pharmaceutical composition according to further embodiments 38 or 39 for treatment of impairment of pancreatic beta-cell function.

Further embodiment 41: A method for lowering autophagy, comprising targeting cells having a receptor associated with their surface that is capable of binding to a chemotactic motif W with a molecule according to any of further embodiments 1 to 30, whereby the molecule is provided with a source of autophagy-inhibiting amino acids selected from the group of alanine (in one letter code: A), glutamine (Q), glycine (G), valine (V), leucine (L), isoleucine (I), proline (P) and arginine (R).

Further embodiment 42: A method for modifying vascular permeability, comprising targeting cells having a receptor associated with their surface that is capable of binding to a chemotactic motif W with a molecule according to any of further embodiments 1 to 30, whereby the molecule is provided with a source of autophagy-inhibiting amino acids selected from the group of alanine (in one letter code: A), glutamine (Q), glycine (G), valine (V), leucine (L), isoleucine (I), proline (P) and arginine (R).

Further embodiment 43: A method for improving tissue repair, comprising targeting cells having a receptor associated with their surface that is capable of binding to a chemotactic motif W with a molecule according to any of further embodiments 1 to 30, whereby the molecule is provided with a source of autophagy-inhibiting amino acids selected from the group of alanine (in one letter code: A), glutamine (Q), glycine (G), valine (V), leucine (L), isoleucine (I), proline (P) and arginine (R).

Further embodiment 44: A method for modulating an immune response, comprising targeting cells having a receptor associated with their surface that is capable of binding to a chemotactic motif W with a molecule according to any of further embodiments 1 to 30, whereby the molecule is provided with a source of autophagy-inhibiting amino acids selected from the group of alanine (in one letter code: A), glutamine (Q), glycine (G), valine (V), leucine (L), isoleucine (I), proline (P) and arginine (R).

Further embodiment 45: A method according to any one of further embodiments 41-44, wherein the receptor is selected from the group of formyl-peptide receptors, complement receptors and CXC-receptors.

Further embodiment 46: A method according to any one of further embodiments 41-45, wherein the chemotactic motif W is selected from a group of motifs, more preferably selected from fMLP, WKYMVm (SEQ ID NO:6), xPGP (SEQ ID NO:16), AcPGP, SGP, AcSGP, YSFKDMQLGR (SEQ ID NO:3) and AcYSFKPMPLaR (SEQ ID NO:2).

Further embodiment 47: A method according to any one of further embodiments 41-46, wherein the molecule has a formyl-peptide receptor binding motif preferably selected from the group fMLF, fMLKLIV (SEQ ID NO:36), fMIVIL(SEQ ID NO:30), fMMYALF (SEQ ID NO:35), fMIVTFL (SEQ ID NO;44), and fMYVKWPWYVWL (SEQ ID NO:45) more preferably represented by fMLF (f is herein standing for N-formyl-).

Further embodiment 48: A method according to any one of further embodiments 41-46, wherein the molecule has a formyl-peptide receptor binding motif preferably selected from the group WKYMVm (wherein small capital m indicates D-methionine) (SEQ ID NO:6), LESIFRSLLFRVM (SEQ ID NO:46), KWPWYVWL (SEQ ID NO:47), KWPWYIWL (SEQ ID NO:48), KWPWWVWL (SEQ ID NO:49), and KWPWWIWL (SEQ ID NO:50), more preferably represented by WKYMVm (SEQ ID NO:6).

Further embodiment 49: A method according to any one of further embodiments 41-46, wherein the molecule has a CXC-receptor binding motif represented by PGP.

Further embodiment 50: A method according to any one of further embodiments 41-46, wherein the molecule has a CXC-receptor binding motif represented by AcPGP.

Further embodiment 51: A method according to any one of further embodiments 41-46, wherein the molecule has a CXC-receptor binding motif represented by SGP.

Further embodiment 52: A method according to any one of further embodiments 41-46, wherein the molecule has a CXC-receptor binding motif represented by AcSGP.

Further embodiment 53: A method according to any one of further embodiments 41-46, wherein the molecule has a C5α-receptor binding motif represented by YSFKDMQLGR (SEQ ID NO:3).

Further embodiment 54: A method according to any one of further embodiments 41-46, wherein the molecule has a C5α-receptor binding motif represented by AcYSFKPMPLaR (SEQ ID NO:2).

Further embodiment 55: A method according to any one of further embodiments 41-54, wherein the source of autophagy-inhibiting amino acids is a peptide comprising the autophagy-inhibiting amino acids.

Further embodiment 56: A method according further embodiment 55, wherein the peptide comprising the autophagy-inhibiting amino acids comprises a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11).

Further embodiment 57: A method according to further embodiments 55 or 56, wherein the chemotactic motif is connected to the peptide comprising the autophagy-inhibiting amino acids by a peptide bond.

Further embodiment 58: A method according to any one of further embodiments 41-46, wherein the molecule capable of binding to a chemotactic motif is a complement-like molecule, preferably selected from C5a fragments or conformationally constrained agonist analogs of C5a.

Further embodiment 59: A method according to any one of further embodiments 41-46, wherein the molecule capable of binding to a chemotactic motif is an antibody-like molecule, preferably selected from IgG, IgM, single chain antibodies, FAB- or FAB′2-fragments.

Further embodiment 60: A method according to further embodiment 58, wherein the source of autophagy-inhibiting amino acids is a peptide comprising a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), connected to the complement-like molecule through a peptide bond.

Further embodiment 61: A method according to further embodiment 60, wherein the complement-like molecule is conjugated to the source of autophagy-inhibiting amino acids.

Further embodiment 62: A method according to further embodiment 59, wherein the source of autophagy-inhibiting amino acids is a peptide comprising a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), connected to the antibody-like molecule through a peptide bond.

Further embodiment 63: A method according to further embodiment 61, wherein the antibody-like molecule is conjugated to the source of autophagy-inhibiting amino acids.

Further embodiment 64: A method according to any one of further embodiments 57-63, wherein the source of autophagy-inhibiting amino acids is a lipid vesicle such as a liposome.

Further embodiment 65: A method according to further embodiment 64, wherein the liposome comprises a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11).

Further embodiment 66: A molecule provided with a chemotactic motif for use in lowering autophagy, the molecule comprising a source of autophagy-inhibiting amino acids, selected from the group of alanine (in one letter code: A), glutamine (Q), glycine (G), valine (V), leucine (L), isoleucine (I), proline (P) and arginine (R).

Further embodiment 67: A molecule provided with a chemotactic motif for use in the modulation of an immune response, the molecule comprising a source of autophagy-inhibiting amino acids, selected from the group of alanine (in one letter code: A), glutamine (Q), glycine (G), valine (V), leucine (L), isoleucine (I), proline (P) and arginine (R).

Further embodiment 68: A molecule provided with a chemotactic motif for use in improving tissue repair, the molecule comprising a source of autophagy-inhibiting amino acids, selected from the group of alanine (in one letter code: A), glutamine (Q), glycine (G), valine (V), leucine (L), isoleucine (I), proline (P) and arginine (R).

Further embodiment 69: A molecule provided with a chemotactic motif for use in modifying vascular permeability, the molecule comprising a source of autophagy-inhibiting amino acids, selected from the group of alanine (in one letter code: A), glutamine (Q), glycine (G), valine (V), leucine (L), isoleucine (I), proline (P) and arginine (R).

Further embodiment 70: A molecule according to any one of further embodiments 66-69, wherein the chemotactic motif is recognized by a receptor selected from the group of formyl-peptide receptors, complement receptors and CXC-receptors.

Further embodiment 71: A molecule according to any one of embodiments 66-69, wherein the molecule has a formyl-peptide receptor binding motif represented by fMLP.

Further embodiment 72: A molecule according to any one of embodiments 66-69, wherein the molecule has a formyl-peptide receptor binding motif represented by WKYMVm (SEQ ID NO:6).

Further embodiment 73: A molecule according to any one of embodiments 66-69, wherein the molecule has a CXC-receptor binding motif represented by PGP.

Further embodiment 74: A molecule according to any one of embodiments 66-69, wherein the molecule has a CXC-receptor binding motif represented by AcPGP.

Further embodiment 75: A molecule according to any one of embodiments 66-69, wherein the molecule has a CXC-receptor binding motif represented by SGP.

Further embodiment 76: A molecule according to any one of embodiments 66-69, wherein the molecule has a CXC-receptor binding motif represented by AcSGP.

Further embodiment 77: A molecule according to any one of embodiments 66-69, wherein the molecule has a C5α-receptor binding motif represented by YSFKDMQLGR (SEQ ID NO:3).

Further embodiment 78: A molecule according to any one of embodiments 66-69, wherein the molecule has a C5α-receptor binding motif represented by AcYSFKPMPLaR (SEQ ID NO:2).

Further embodiment 79: A molecule according to any one of embodiments 66-69, wherein the molecule capable of binding to a chemotactic motif is a complement-like molecule, preferably selected from C5a fragments or conformationally constrained agonist analogs of C5a.

Further embodiment 80: A molecule according to any one of embodiments 66-69, wherein the molecule capable of binding to a chemotactic motif is an antibody-like molecule, selected from IgG, IgM, single chain antibodies, FAB- or FAB′2-fragments.

Further embodiment 81: A molecule according to any one of embodiments 66-80, wherein the source of autophagy-inhibiting amino acids is a peptide comprising a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11).

Further embodiment 82: A molecule according to embodiment 81, wherein the molecule is connected to the peptide through a peptide bond.

Further embodiment 83: A peptide provided with a chemotactic motif for use in lowering autophagy, the peptide comprising a source of autophagy-inhibiting amino acids, selected from the group of alanine (in one letter code: A), glutamine (Q), glycine (G), valine (V), leucine (L), isoleucine (I), proline (P) and arginine (R).

Further embodiment 84: A peptide provided with a chemotactic motif for use in the modulation of an immune response, the peptide comprising a source of autophagy-inhibiting amino acids, selected from the group of alanine (in one letter code: A), glutamine (Q), glycine (G), valine (V), leucine (L), isoleucine (I), proline (P) and arginine (R).

Further embodiment 85: A peptide provided with a chemotactic motif for use in improving tissue repair, the peptide comprising a source of autophagy-inhibiting amino acids, selected from the group of alanine (in one letter code: A), glutamine (Q), glycine (G), valine (V), leucine (L), isoleucine (I), proline (P) and arginine (R).

Further embodiment 86: A peptide provided with a chemotactic motif for use in modifying vascular permeability, the peptide comprising a source of autophagy-inhibiting amino acids, selected from the group of alanine (in one letter code: A), glutamine (Q), glycine (G), valine (V), leucine (L), isoleucine (I), proline (P) and arginine (R).

Further embodiment 87: A peptide according to any one of embodiments 83-86, wherein the chemotactic motif is recognized by a receptor selected from the group of formyl-peptide receptors, complement receptors and CXC-receptors.

Further embodiment 88: A peptide according to any one of embodiments 83-86, wherein the peptide has a formyl-peptide receptor binding motif represented by fMLP.

Further embodiment 89: A peptide according to any one of embodiments 83-86, wherein the peptide has a formyl-peptide receptor binding motif represented by WKYMVm (SEQ ID NO:6).

Further embodiment 90: A peptide according to any one of embodiments 83-86, wherein the peptide has a CXC-receptor binding motif represented by PGP.

Further embodiment 91: A peptide according to any one of embodiments 83-86, wherein the peptide has a CXC-receptor binding motif represented by AcPGP.

Further embodiment 92: A peptide according to any one of embodiments 83-86, wherein the peptide has a CXC-receptor binding motif represented by SGP.

Further embodiment 93: A peptide according to any one of embodiments 83-86, wherein the peptide has a CXC-receptor binding motif represented by AcSGP.

Further embodiment 94: A peptide according to any one of embodiments 83-86, wherein the peptide has a C5α-receptor binding motif represented by YSFKDMQLGR (SEQ ID NO:3).

Further embodiment 95: A peptide according to any one of embodiments 83-86, wherein the peptide has a C5α-receptor binding motif represented by AcYSFKPMPLaR (SEQ ID NO:2).

Further embodiment 96: A peptide according to any one of embodiments 83-95, comprising a peptide selected from a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), for use in lowering autophagy.

Further embodiment 97: A peptide comprising at least 4 amino acids and at most 30 amino acids comprising a sequence of the formula φnW, or Wφn, or φWφm wherein W represents a chemotactic motif, φ is an autophagy-inhibiting amino acid and n=an integer from 1 to 24 and m is an integer from 1-23, whereby n+m is no greater than 24.

Further embodiment 98: A peptide according to embodiment 97, wherein W represents a chemotactic motif recognized by a receptor selected from the group of formyl-peptide receptors, complement receptors and CXC-receptors.

Further embodiment 99: A peptide according to embodiment 97 or 98, wherein III is selected from a group of motifs represented by fMLP, WKYMVm (SEQ ID NO:6), PGP, AcPGP, SGP, AcSGP, YSFKDMQLGR (SEQ ID NO:3) and AcYSFKPMPLaR (SEQ ID NO:2).

Further embodiment 100: A peptide according to any one of embodiments 97-99, wherein φ is selected from the group of autophagy-inhibiting amino acids alanine (in one letter code: A), glutamine (Q), glycine (G), valine (V), leucine (L), isoleucine (I), proline (P) and arginine (R).

Further embodiment 101: A peptide according any one of embodiments 97-100, wherein φn and/or φm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11).

Further embodiment 102: A pharmaceutical formulation comprising a peptide according to embodiments 97-101 and at least one pharmaceutically acceptable excipient.

Further embodiment 103: A pharmaceutical formulation comprising a peptide comprising a peptide according to embodiments 97-102 and a peptide selected from a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group LQGV (SEQ ID NO:10) and AQGV (SEQ ID NO:11), for use in lowering autophagy, and at least one pharmaceutically acceptable excipient.

Further embodiment 104: A method for producing a peptide according to embodiments 97-101 comprising synthesizing the peptide with an automated peptide synthesizer.

Further embodiment 105: A peptide according to embodiments 97-101 obtainable with a method according to embodiment 104 for use in a method selected from the group of lowering autophagy, modifying vascular permeability, improving tissue repair and modulating an immune response.

### Autophagy-Inhibiting Peptides (AIPs)

A peptide is a chain of amino acids in which the α-amino group of one amino acid is bonded to the α-carboxyl group of the next. Thus, each bond linking the amino acids is a secondary amide, called a peptide bond. If a peptide made from two amino acids is a dipeptide, one made from three is a tripeptide, and so forth. As we have seen many times, the prefixes, di-, tri-, tetra-, etc., indicate the number of amino acid units from which the chain is made. Peptides that contain only a few amino acids up to about fifty are called oligopeptides; peptides with more than 50 amino acids are called polypeptides, a term synonymous with protein.

A peptide has two ends: the end with a free amino group is called the N-terminal amino acid residue. The end with a free carboxyl group is called the C-terminal amino acid residue. Peptides are named from the N-terminal acid residue to the C-terminal amino acid. Amino acid sequences within a (poly)peptide are herein also identified as peptide. In describing protein or peptide composition, structure and function herein, reference is made to amino acids. In the present specification, amino acid residues are expressed by using the following abbreviations. Also, unless explicitly otherwise indicated, the amino acid sequences of peptides and proteins are identified from N-terminal to C-terminal, left terminal to right terminal, the N-terminal being identified as a first residue. Ala: alanine residue; Asp: aspartate residue; Glu: glutamate residue; Phe: phenylalanine residue; Gly: glycine residue; His: histidine residue; Ile: isoleucine residue; Lys: lysine residue; Leu: leucine residue; Met: methionine residue; Asn: asparagine residue; Pro: proline residue; Gln: glutamine residue; Arg: arginine residue; Ser: serine residue; Thr: threonine residue; Val: valine residue; Trp: tryptophan residue; Tyr: tyrosine residue; Cys: cysteine residue. The amino acids may also be referred to by their conventional one-letter code abbreviations; A=Ala; T=Thr; V=Val; C=Cys; L=Leu; Y=Tyr; I=Ile; N=Asn; P=Pro; Q=Gln; F=Phe; D=Asp; W=Trp; E=Glu; M=Met; K=Lys; G=Gly; R=Arg; S=Ser; and H=His.

“Peptide” shall mean herein a natural biological or artificially manufactured (synthetic) short chain of amino acid monomers linked by peptide (amide) bonds. Glutamine peptide shall mean herein a natural biological or artificially manufactured (synthetic) short chain of amino acid monomers linked by peptide (amide) bonds wherein one of the amino acid monomers is a glutamine.

Chemically synthesized peptides generally have free N- and C-termini. N-terminal acetylation and C-terminal amidation reduce the overall charge of a peptide; therefore, its overall solubility might decrease. However, the stability of the peptide could also be increased because the terminal acetylation/amidation generates a closer mimic of the native protein. These modifications might increase the biological activity of a peptide and are herein also provided.

### Peptide Synthesis

Peptides or retro-inverso variants thereof are synthesized according to classical solid phase synthesis. Purity of the peptides is confirmed by high performance liquid chromatography and by fast atom bombardment mass spectrometry. Traditionally, peptides are defined as molecules that consist of between 2 and 50 amino acids, whereas proteins are made up of 50 or more amino acids. In addition, peptides tend to be less well defined in structure than proteins, which can adopt complex conformations known as secondary, tertiary, and quaternary structures. Functional distinctions may also be made between peptides and proteins. In fact, most researchers, as well as this application, use the term “peptide” to refer specifically to peptides, or otherwise relatively short amino acid chains of up to 50 amino acids (also called oligopeptides), with the term “polypeptide” being used to describe proteins, or chains of >50 or much more amino acids.

### Determination of Chemotactic Activity.

Human U937 monocytic cells are purchased from the American Type Culture Collection (ATCC catalog number CRL-1593.2, Manassas, Va). Cells are maintained in suspension culture in T-75 flasks containing RPMI 1640 medium supplemented with 10% fetal calf serum and antibiotics, and cultures are split every 3 to 5 days. Three days before use in chemotaxis assays, U937 cells are stimulated to differentiate along the macrophage lineage by exposure to 1 mmol/L dibutyryl cyclic adenosine monophosphate (dbcAMP; Sigma Chemical Co), as described. Cells are washed three times to remove culture medium and then resuspended in chemotaxis medium (Dulbecco's modified essential medium supplemented with 1% lactalbumin hydrolysate) for plating into assay chambers at a final concentration of 2.5×106 cells/mL. Chemotaxis assays are performed in 48-well microchemotaxis chambers (Neuro Probe, Cabin John, Md). The bottom wells of the chamber are filled with 25 mL of the chemotactic stimulus (or medium alone) in triplicate. An uncoated 10-mm-thick polyvinylpyrrolidone-free polycarbonate filter with a pore size of 5 mm is placed over the samples (Neuro Probe). The silicon gasket and the upper pieces of the chamber are applied, and 50 mL of the monocyte cell suspension are placed into the upper wells. Chambers are incubated in a humidified 5% CO2 atmosphere for 3 hours at 37° C., and nonmigrated cells are gently wiped away from the upper surface of the filter. The filter is immersed for 30 seconds in a methanol-based fixative and stained with a modified Wright-Giemsa technique (Protocol Hema 3 stain set; Biochemical Sciences, Inc, Swedesboro, NJ) and then mounted on a glass slide. Cells that are completely migrated through the filter are counted under light microscopy, with 3 random high-power fields (HPF; original magnification ×400) counted per well.

Human monocytes are isolated from freshly drawn blood of healthy volunteers using serial Ficoll/PCXC receptor (ERC)oll gradient centrifugation, as described elsewhere. Cells are cultured for 16 hours in RPMI-1640 media supplemented with 0.5% human serum to become quiescent after isolation. Purity of the cells is >95% as determined by flow cytometry analysis. Monocyte chemotaxis is assayed in a 48-well microchemotaxis chamber (Neuroprobe, Gaithersburg, MD) in serum-free media. Wells in the upper and lower chamber are separated by a polyvinylpyrrolidone-free polycarbonate membrane (pore size 5 m; Costar). Freshly isolated monocytes at a density of 5×105/mL are incubated for 2.5 hours with recombinant C-peptide (Sigma), before migrated cells on the bottom face of the filter are stained and counted under the light microscope. Maximal chemotactic activity is measured with 0.1 mmol/L N-formyl-methionyl-leucyl-phenylalanine (f-MLF; Sigma Chemical Co), and checkerboard analysis is used to distinguish chemotaxis from chemokinesis.

Chemotaxis is also assayed by a double micropore membrane system in modified Boyden chambers. The lower compartment containing 180 μl of peptide or fragments thereof at various concentrations is separated from the upper compartment containing 200 μl of cell suspension (5≠104 cells, such as endothelial cells or smooth muscle cells or pericytes or keratinocytes of fibroblasts or leukocytes per ml medium) by a 10 μm polycarbonate membrane (Millipore, Bedford, MA). The membranes are presoaked in bovine type I collagen (25 micro-g phosphate-buffered saline per ml) (Chemicon International, Temecula, CA) for 24 hours at room temperature to facilitate the attachment of cells. The chambers are incubated for 18 hours at 37° C. in 5% C02-balanced air. The chambers are then disassembled and the membrane pairs are stained with hematoxylin. The cell number of a number, such as five, random and non-overlapping fields under a microscope is counted. Chemotaxis is assayed as described above. Chemotaxis may also be studied in an ex vivo aortic ring assay measuring endothelial cell migration and proliferation.

### Cell Isolation

Blood is drawn from healthy volunteers into tubes containing citrate as an anticoagulant. Neutrophils are isolated by using a Polymorphprep kit (Nicomed, Oslo, Norway) according to the manufacturer's instructions; monocytes are purified with magnetic beads (Miltenyi Biotech). The purity of the cells, as assessed by flow cytometry (anti-CD45, 14, DR, and CD66b), is >93%. For each cell type, samples from two different donors are examined.

Distribution and Elimination of Intravenously Injected [14C]-AQGV (SEQ ID NO:11) from Mice

The present study, which was conducted by TNO Biosciences, Utrechtseweg, Netherlands, was designed to provide data on the distribution and metabolism of [14C]-AQGV (SEQ ID NO:11) (Ala-Gln-[1-¹⁴C]Gly-Val) in male CD-1 mice following a single intravenous dose of 50 mg/kg. To this end, mice were sacrificed at 10, 30 and 60 minutes, and 6 and 24 hours after administration of radiolabeled AQGV (SEQ ID NO:11), counts in various tissues were determined, and the radioactivity present in the urine and plasma were analyzed by HPLC.

There was relatively little radioactivity in the blood 10 minutes after the radiolabeled peptide was injected. If all of the counts were in intact peptide, the amount present would be 17.2 μg/g. No parent compound could be detected in plasma after 10 minutes; however, [¹⁴C]-AQGV (SEQ ID NO:11) appeared to be hydrolyzed quite rapidly. No parent compound was detected in the urine either. The radioactivity in urine was mostly present as hydrophilic compounds, eluting in or just after the dead volume of the HPLC column.

The radioactivity present in various organs exceeded that in the blood. The highest concentrations of “peptide” after 10 minutes were found in the kidneys (362 μg/g), liver (105 μg/g), testis (85.7 μg/g), lung (75.2 μg/g), and spleen (74.7 μg/g). In general, a gradual decrease in radioactivity was observed thereafter. After 24 hours, the highest concentrations were found in kidneys (61.9 μg/g), thymus (43.1 μg/g), spleen (39.3 μg/g), liver (37.6 μg/g), and skin (37.5 μg/g).

The average total recoveries of radioactivity 10, 30, and 60 minutes, and 6 and 24 hours after dose administration were 83.2, 70.5, 62.9, 52.6 and 50.8%, respectively. These results strongly indicate that there is rapid formation of [14C]-volatiles after dose administration, most likely ¹⁴C-CO2, which is exhaled via the expired air. After 24 hours, 10.2% of the administered radioactivity was excreted in urine, and 2.6% in feces.

### Conclusions

After intravenous injections, [¹⁴C]-AQGV (SEQ ID NO:11) was rapidly removed from the blood. This is consistent with the results of pharmacokinetic studies that are presented below. Metabolite profiles in blood plasma and urine revealed no parent compound, indicating rapid metabolism of [¹⁴C]-AQGV (SEQ ID NO:11). About 50% of the administered radioactivity was exhaled as volatiles, most likely ¹⁴C-CO2, up to 24 hours. The results of the present study indicate rapid hydrolysis of [¹⁴C]-AQGV (SEQ ID NO:11) yielding [1-¹⁴C]-glycine, which is subsequently metabolized into ¹⁴C-CO2 and exhaled in the expired air. The absence of parent compound in plasma and urine suggests that the radioactivity present in tissues and organs could be present only as hydrolyzation products of the metabolism of [¹⁴C]-AQGV (SEQ ID NO:11).

### Peptide Hydrolysis

The disclosure provides that when a peptide provided with autophagy-inhibiting amino acids, such as comprising a peptide AQGV (SEQ ID NO:11), encounters a cell, the peptide is hydrolyzed, be it extracellular at the surface of that cell, or after endocytosis, in the case of vascular cells, for example, by elastin receptor-mediated endocytosis, of the peptide by the cell in the phagolysosome. Many peptidases are known to exist on or in cells that can rapidly hydrolyze peptides, and continued hydrolysis invariably leads to tripeptides and dipeptides. Likewise, hydrolysis in the lysosomes by tripeptidyl and dipeptidyl peptidase will equally result in single amino acids. Studies with ¹⁴C labeled AQGV (SEQ ID NO:11) have factually shown full hydrolysis of the peptide in 15 minutes after its administration in mice. Tripeptides, dipeptides and single amino acids will result from the hydrolysis of AQGV (SEQ ID NO:11), or its sister compound LQGV (SEQ ID NO:10), or for that matter from any other suitable oligopeptide, when presented to a cell.

### Peptide Transport

Similarly, several studies have reported the role of p38 MAPK in survival of different type of mature granulocytes. Granulocytes (e.g., neutrophils, eosinophils, basophils) have in common their terminal differentiation stage. These cells have fragmented nuclei and have an accumulation of granules containing preformed secretion factors. It is herein been proposed that p38 MAPK is required for survival of neutrophils, and inactivation of p38 MAPK is essential for death and the elimination of these cells as well as that p38 MAPK is required for contraction of endothelial cells, and inactivation of p38 MAPK is essential for relaxing those vascular cells so that those can restore vascular wall integrity, as well as inactivation of p38 MAPK activity is essential for pacifying neutrophils, and other leucocytes cells exploring the vascular permeability of vascular endothelial blood vessel wall.

Di- and tripeptides are selectively transported via the PEPT1/2 transporters. Tripeptides, dipeptides and single amino acids are actively transported through the cell membrane, whereby uptake of dipeptides and tripeptides involves a separate mechanism than uptake of single amino acids, namely via the PEPT1 and PEPT2 transporters. Potentially all 400 di- and 8,000 tripeptides can be transported by PepTI and PEPT2. Intestinal cell transport of amino acids in the form of peptides was demonstrated to be a faster route of uptake per unit of time than their constituent amino acids in the free form (reviewed in J. Anim. Sci., 2008; 9, 2135-2155).

mTOR is Involved

Finally, we propose involvement of mechanistic target of rapamycine, mTOR. In this perspective, the peptide enters cells either via PEPT1/2 or by active endocytosing or phagocytosing processing, after which the peptide is fully hydrolyzed in the phagolysosome and the resulting autophagy-inhibiting amino acids are presented to mTOR complex where they cause inhibition of autophagy of the cell. Tetrapeptide, tripeptide and dipeptide activities may all reflect the final causal activity of single amino acids A, Q, G, V, selected from the group of amino acids A,Q,G,V,L and P. In this way, the amino acids A,Q,G,V,L and P, are food for mTOR. Indeed, preliminary results show similar effects on inhibition of the p38 pathway when different tri- and dipeptides derived from AQGV (SEQ ID NO:11) in an FPR-signaling assay are used. Individual amino acids may approach mTOR via the cytosol, but amino acids in peptide fragments (strings such as AQGV (SEQ ID NO:11)), must enter the mTOR machinery via the phagolysosome. Activation of mTOR by amino acids can, therefore, be explained from two perspectives, 1) whereby endocytosis of peptide strings is paramount for all phagocytosing cells, such as neutrophils and monocytes, 2) whereby peptide fragments enter via PEPT1/2.

Amino Acids Activate mTOR Pathways and Inhibit Autophagy

Autophagy serves to produce amino acids for the survival of a cell when nutrients fall short, and amino acids are effective inhibitors of autophagy. Mechanistic-target-of-rapamicin (mTOR) is a critical regulator of autophagy induction, with activated mTOR suppressing autophagy and negative regulation of mTOR promoting it. Amino acids are indeed considered important regulators of mTOR complex 1 or 2 activation, affecting cell proliferation, protein synthesis, autophagy and survival. These findings identify new signaling pathways used by amino acids underscoring the crucial importance of these nutrients in cell metabolism and offering new mechanistic insights in developing pharmaceutically active peptides.

Differential Signaling of Amino Acids to the mTOR Pathway.

Some amino acids are known to control proteogenesis (mTOR kinases) or proteolysis (autophagy) more than others. Recent and older data (literature search November 2011) identify leucine (L), valine (V), isoleucine (I), alanine (A), glutamine (Q), arginine (R), glycine (G), proline (P), either alone or in combination, as more potent activators of mTOR or inhibitors of autophagy than other amino acids, such as glutamate (E), threonine (T), serine (S), lysine (K), threonine (T), phenylalanine (F), tyrosine (Y), and methionine (M) that have been reported to have no or opposite effects. Amino acids leucine (L), alanine (A), glutamine (Q), and proline (P) are reported to have most prominent inhibitory effects on autophagy in human cells (A. J. Meijer et al., Amino Acids 2015, 47, 2037-2063).

TABLE 2

| Literature-based activity | | | mTOR kinases | autophagy | |
|---|---|---|---|---|---|
| L-Glycine | Gly | G | | DOWN | Bluem J. Biol. Chem. 2007 37783 |
| | | | | DOWN | Qin et al. http://en.cnki.com.cn/Article_en/CJFDTOTAL- |
| | | | | | NJYK200912002.htm |
| L-Alanine | Ala | A | | DOWN | Proc. Natl. Acad. Sci. USA Vol. 76, No. 7, pp. 3169-3173, |
| | | | | | July 1979 |
| L-Proline | Pro | P | activate | | Washington Am. J. Physiol. Cell Physiol. 2010 298 C982 |
| L-Valine | Val | V | activate | | Maria Dolors Sans et al., J. Nutr. 136: 1792-1799, 2006. |
| L-Isoleucine | Ile | I | activate | DOWN | Doi J. Nutr. 135 2102-8 Biochem. Biophys. Acta 2008 |
| | | | | | 1115 |
| L-Isoleucine | | | | | Maria Dolors Sans et al., J. Nutr. 136: 1792-1799, 2006. |
| L-Leucine | Leu | L | activate | DOWN | Ijichi et al., Bioc. Biophys. Res. Com. 2003 303 59 |
| L-Leucine | | | | | Maria Dolors Sans et al., J. Nutr. 136: 1792-1799, 2006. |
| L-Glutamine | Gln | Q | activate | DOWN | Amino Acids 2009 73 111 |
| L-Glutamine | | | activate | DOWN | Kim Biol. Reprod. 2011 84 1139 |
| L-Arginine | Arg | R | activate | DOWN | Ban et al., Int. J. Mol. Med. 2004 13: 537-43 |
| L-Arginine | | | activate | DOWN | Kim Biol. Reprod. 2011 84 79 |
| L-Tyrosine | Tyr | Y | | | |
| L-Lysine | Lys | K | down | | Prizant J. Cell. Biochem. 2008 1 1038 |
| L-Tryptophan | Trp | W | | | |
| L-Cystine | Cys | C | | | |
| L-Serine | Ser | S | | | |
| L-Threonine | Thr | T | down | | Prizant J. Cell. Biochem. 2008 1 1038 |
| L-Asparagine | Asn | N | | | |
| L-Aspartic acid | Asp | D | | | |
| L-Methionine | Met | M | partial inhibit | | Stubs J. Endocrinol. 2002 174 335 |
| L-Histidine | His | H | down | | Prizant J. Cell Biochem. 2008 1 1038 |
| L-Phenylalanine | Phe | F | | | |
| L-Glutamic Acid | Glu | E | | | |

Peptides Enriched with Amino Acids that Inhibit Autophagy

Examples of autophagy-inhibiting-peptides (ATPs) that are enriched with these above amino acids and down-regulate disease are, for example, dipeptide AQ, QQ, LQ, GQ, PQ, VQ, AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, QOLG, QGG, QPG, QVG, LAG, LGG, LPG, LVG or a tetrapeptide AQGV (SEQ ID NO:11), QQGV (SEQ ID NO:51), LQGV (SEQ ID NO:10), GQGV (SEQ ID NO:53), PQGV (SEQ ID NO:54), VQGV (SEQ ID NO:55), ALGV (SEQ ID NO:56), LLGV (SEQ ID NO:57), QLGV (SEQ ID NO:58), GLGV (SEQ ID NO:59), PLGV (SEQ ID NO: 60), VLGV (SEQ ID NO:61), QAGV (SEQ ID NO:62), QGGV (SEQ ID NO:64), QPGV (SEQ ID NO:65), QVGV (SEQ ID NO:66), LAGV (SEQ ID NO:67), LGGV (SEQ ID NO:68), LPGV (SEQ ID NO:69), LVGV (SEQ ID NO:70), and mixtures thereof, such as AQ+GV, and AQ+VG, and LQ+GV, and LQ+VG, which are herein provided as selected from the group of dipeptide AQ, QQ, LQ, GQ, PQ, VQ, AL, LL, QL, GL, PL, VL, QA, QL, QG, QP, QV, LA, LG, LP, LV, a tripeptide AQG, QQG, LQG, GQG, PQG, VQG, ALG, LLG, QLG, GLG, PLG, VLG, QAG, QOLG, QGG, QPG, QVG, LAG, LGG, LPG, LVG or a tetrapeptide AQGV (SEQ ID NO:11), QQGV (SEQ ID NO:51), LQGV (SEQ ID NO:10), GQGV (SEQ ID NO:53), PQGV (SEQ ID NO:54), VQGV (SEQ ID NO:55), ALGV (SEQ ID NO:56), LLGV (SEQ ID NO:57), QLGV (SEQ ID NO:58), GLGV (SEQ ID NO:59), PLGV (SEQ ID NO:60), VLGV (SEQ ID NO:61), QAGV (SEQ ID NO:62), QGGV (SEQ ID NO:64), QPGV (SEQ ID NO:65), QVGV (SEQ ID NO:66), LAGV (SEQ ID NO:67), LGGV (SEQ ID NO:68), LPGV (SEQ ID NO:69), LVGV (SEQ ID NO:70), or a mixture thereof. Other peptides are now easily derived, preferably by generating or synthesizing small peptides by combining amino acids that preferentially activate mTOR or preferentially inhibit autophagy, preferably selected from the group of A, G, L, V, Q and P, into strings of peptides.

### Assumed Mode of Action

Administered peptide or amino acid fragments thereof are, for example, taken up by amino acid transport, PEPT1/2 transport, by common endocytosis, in the case of vascular cells by elastin receptor-mediated endocytosis or by common phagocytosis. Internalized peptide is hydrolyzed and its amino acids are presented to the nutrient-sensing system of mTOR. As it now herein emerges, these peptides preferably need be hydrolyzed into individual amino acids before they can act at the nutrient-sensing-system of mTOR, thus it can be understood why receptor meditated activity has never unequivocally been demonstrated. As to routing into the cell, most di- and tripeptides are readily taken up by PEPT1/2 transporters present in intestinal epithelial cells, renal tubular cells and other cells. Also, tetra- to hexapeptide uptake is regularly achieved by common endocytosis, in the case of vascular cells by elastin receptor-mediated endocytosis, allowing targeting cells for uptake by phagocytosis. Internalized peptide is hydrolyzed and its amino acids are presented to the nutrient-sensing system of mTOR. Considering the broad mode-of-action here displayed, the tissue-repair signal molecule peptides provided in the disclosure can advantageously be used in combined treatment with most biologic therapies.

### Rational Design

A great advantage of this new class of autophagy-inhibiting peptides (herein also indicated as autophagy-inhibiting molecules) is that they are easily synthesized, stabilized and modified, the main requirement being that they comprise amino acids that target the nutrient sensing system of mTOR and preferentially inhibit autophagy.

The disclosure also provides synthetic peptides wherein any one peptide with chemoattractant motif W comprising AIPs has been repeated at least once, optionally, the repeats are separated by a linker, such a linker may comprise one or more amino acids, such as one or more amino acids selected from the group of glycine, alanine, leucine, valine, isoleucine or glutamine.

### Pharmaceutical Compositions

### Example 1

Peptide
(SEQ ID NO: 71)
KWPWYIWLAQGV

To prepare 10 ml of the composition, mix:
Peptide (KWPWYIWLAQGV (SEQ ID NO:71))--500 mg
M-Kreosol--25 mg
Glycerol--160 mg
Water and either 10% hydrochloric acid or 10% sodium hydroxide sufficient to make a composition volume of 10 ml and a final pH of 7.0-7.8. Optionally, an acid-resistant capsule is filled with above composition.

### Example 2

Peptide
(SEQ ID NO: 72)
KWPWYIWLAQLPGP

To prepare 10 ml of the composition, mix:
Peptide (KWPWYIWLAQLPGP (SEQ ID NO:72))--500 mg
M-Kreosol--25 mg
Glycerol--160 mg
Water and either 10% hydrochloric acid or 10% sodium hydroxide sufficient to make a composition volume of 10 ml and a final pH of 7.0-7.8. Optionally, an acid-resistant capsule is filled with above composition.

### Example 3

Peptide
(SEQ ID NO: 73)
KWPWYIWLQGVLPALP

To prepare 10 ml of the composition, mix:
Peptide (KWPWYIWLQGVLPALP (SEQ ID NO:73))--500 mg
M-Kreosol--25 mg
Glycerol--160 mg
Water and either 10% hydrochloric acid or 10% sodium hydroxide sufficient to make a composition volume of 10 ml and a final pH of 7.0-7.8. Optionally, an acid-resistant capsule is filled with above composition.

### Example 4

Peptide
(SEQ ID NO: 74)
KWPWYIWLQGLQPGQ

To prepare 10 ml of the composition, mix:
Peptide (KWPWYIWLQGLQPGQ (SEQ ID NO:74))--500 mg
M-Kreosol--25 mg
Glycerol--160 mg
Water and either 10% hydrochloric acid or 10% sodium hydroxide sufficient to make a composition volume of 10 ml and a final pH of 7.0-7.8. Optionally, an acid-resistant capsule is filled with above composition.

### Example 5

Peptide WPWYIWLQGLQPGQ (SEQ ID NO:75) and insulin

To prepare 10 ml of the composition, mix:
Human Insulin (28 U/mg)--1000 U
Peptide 1 (WPWYIWLQGLQPGQ (SEQ ID NO:75))--500 mg
M-Kreosol--25 mg
Glycerol--160 mg
Water and either 10% hydrochloric acid or 10% sodium hydroxide sufficient to make a composition volume of 10 ml and a final pH of 7.0-7.8.

### Example 6

Peptide fMLFLQGLQPGQ (SEQ ID NO:76) and insulin

To prepare 10 ml of the composition, mix:
Human Insulin (28 U/mg)--1000 U
Peptide (fMLFLQGLQPGQ (SEQ ID NO:76))--500 mg
M-Kreosol--25 mg
Glycerol--160 mg
Water and either 10% hydrochloric acid or 10% sodium hydroxide sufficient to make a composition volume of 10 ml and a final pH of 7.0-7.8.

### Example 7

Peptide AcPGPAQGLQPGQ (SEQ ID NO:77) and insulin

To prepare 10 ml of the composition, mix:
Human Insulin (28 U/mg)--1000 U
Peptide (AcPGPAQGLQPGQ (SEQ ID NO:77))--500 mg
M-Kreosol--25 mg
Glycerol--160 mg
Water and either 10% hydrochloric acid or 10% sodium hydroxide sufficient to make a composition volume of 10 ml and a final pH of 7.0-7.8.

### Example 8

Peptide AcPGPLQGLQPGQ (SEQ ID NO:78) and insulin

To prepare 10 ml of the composition, mix:
Human Insulin (28 U/mg)--1000 U
Peptide (AcPGPLQGLQPGQ (SEQ ID NO:78))--500 mg
M-Kreosol--25 mg
Glycerol--160 mg
Water and either 10% hydrochloric acid or 10% sodium hydroxide sufficient to make a composition volume of 10 ml and a final pH of 7.0-7.8.

### Example 9

Peptide
(SEQ ID NO: 79)
AcPGPAQGVAPGP

To prepare 10 ml of the composition, mix:
Peptide (AcPGPAQGVAPGP (SEQ ID NO:79)) 40 mg
PBS or 0.9% NaCi sufficient to make a composition volume of 10 ml.

### Example 10

Peptide
(SEQ ID NO: 80)
fMLFLQGVAPGP

To prepare 10 ml of the composition, mix:
Peptide (fMLFLQGVAPGP (SEQ ID NO:80)) 40 mg
PBS or 0.9% NaCl sufficient to make a composition volume of 10 ml.

### Example 11

Peptide
(SEQ ID NO: 81)
AcYSFKPMPLaRAQGVLPG

To prepare 10 ml of the composition, mix:
Peptide (AcYSFKPMPLaRAQGVLPG (SEQ ID NO:81)) 40 mg
PBS or 0.9% NaCl sufficient to make a composition volume of 10 ml.

### Example 12

Peptide
(SEQ ID NO: 82)
AcYSFKPMPLaRAQGVLPGLQGVLPG

To prepare 10 ml of the composition, mix:
Peptide (AcYSFKPMPLaRAQGVLPGLQGVLPG (SEQ ID NO:82)) 40 mg
PBS or 0.9% NaCl sufficient to make a composition volume of 10 ml.

1-29. (canceled)

30. A method of lowering autophagy in a neutrophil cell, the method comprising:
targeting a neutrophil cell having a receptor associated with the cell's surface that is capable of binding to a chemotactic motif W by providing the cell with a molecule containing the chemotactic motif W, wherein the molecule further comprises a source of autophagy inhibiting amino acids selected from the group consisting of alanine (in one letter code: A), glutamine (Q), glycine (G), valine (V), leucine (L), isoleucine (I), proline (P) and arginine (R).

31. The method according to claim 30, wherein the receptor is selected from the group consisting of a formyl-peptide receptor, a complement receptor, and a CXC-receptor.

32. The method according to claim 30, wherein the chemotactic motif W is selected from the group of motifs consisting of fMLP, WKYMVm (SEQ ID NO:6), xPGP (SEQ ID NO:16), AcPGP, SGP, AcSGP, YSFKDMQLGR (SEQ ID NO:3), and AcYSFKPMPLaR (SEQ ID NO:2).

33. The method according to claim 30, wherein the source of autophagy inhibiting amino acids is an autophagy-inhibiting peptide (AIP) comprising said autophagy inhibiting amino acids.

34. The method according to claim 33, wherein the peptide comprising the autophagy inhibiting amino acids comprise:
a dipeptide selected from the group consisting of AQ, LQ, PQ, VQ, and GQ;
a tripeptide selected from the group consisting of AQL, LQL, PQL, VQL, GQL, PLQ, LQG, PQV, VGQ, LQP, LQV, AQG, QPL, PQV, VGQ and GQG; or
a tetrapeptide selected from LQGV (SEQ ID NO:10) and AQGV (SEQ ID NO:11).

35. The method according to claim 30, wherein the molecule containing the chemotactic motif W is connected to a peptide comprising the autophagy inhibiting amino acids by a peptide bond.

36. The method according to claim 30, wherein the molecule containing the chemotactic motif is a complement-like molecule.

37. The method according to claim 30, wherein the molecule containing the chemotactic motif is an antibody-like molecule.

38. The method according to claim 36, wherein the source of autophagy inhibiting amino acids is a peptide comprising:
a dipeptide selected from the group consisting of AQ, LQ, PQ, VQ and GQ;
a tripeptide selected from the group consisting of AQL, LQL, PQL, VQL, GQL, PLQ, LQG, PQV, VGQ, LQP, LQV, AQG, QPL, PQV, VGQ, and GQG; or
a tetrapeptide selected from LQGV (SEQ ID NO:10) and AQGV (SEQ ID NO:11), connected to the complement-like molecule through a peptide bond.

39. The method according to claim 36, wherein the complement-like molecule is conjugated to the source of autophagy inhibiting amino acids.

40. The method according to claim 37, wherein the source of autophagy inhibiting amino acids is a peptide comprising:
a dipeptide selected from the group consisting of AQ, LQ, PQ, VQ, and GQ;
a tripeptide selected from the group consisting of AQL, LQL, PQL, VQL, GQL, PLQ, LQG, PQV, VGQ, LQP, LQV, AQG, QPL, PQV, VGQ and GQG; or
a tetrapeptide selected from LQGV (SEQ ID NO:10) and AQGV (SEQ ID NO:11),
connected to the antibody-like molecule through a peptide bond.

41. The method according to claim 40, wherein the antibody-like molecule is conjugated to the source of autophagy inhibiting amino acids.

42. The method according to claim 36, wherein the source of autophagy inhibiting amino acids is a lipid vesicle.

43. The method according to claim 30, wherein the autophagy inhibiting amino acids are selected from the group consisting of A, Q, G, L, and P.

44. The method according to claim 43, wherein the autophagy inhibiting amino acids are selected from the group consisting of A, Q, L, and P.

45. A peptide of at least four amino acids and at most 30 amino acids, the peptide comprising:
a sequence of the formula ϕnW, or Wϕn, or ϕWϕm,
wherein
W represents a chemotactic motif;
ϕis an autophagy inhibiting amino acid selected from the group consisting of alanine (in one letter code: alanine A), glutamine (Q), glycine (G), valine (V), leucine (L), isoleucine (I), proline (P), and arginine (R);
n=an integer from 1 to 24; and
m is an integer from 1-23, and
wherein n+m is no greater than 24.

46. The peptide of claim 45, wherein W represents a chemotactic motif recognized by a receptor selected from the group consisting of a formyl-peptide receptor, a complement receptor, and a CXC-receptor.

47. The peptide of claim 45, wherein W is selected from the group of motifs consisting of fMLP, WKYMVm (SEQ ID NO:6), PGP, AcPGP, SGP, AcSGP, YSFKDMQLGR (SEQ ID NO:3), and AcYSFKPMPLaR (SEQ ID NO:2).

48. The peptide of claim 45, wherein 4 is selected from the group consisting of A, Q, G, L, and P.

49. The peptide of claim 45, wherein ϕn and/or ϕm comprise:
a dipeptide selected from the group AQ, LQ, PQ, VQ, and GQ;
a tripeptide selected from the group AQL, LQL, PQL, VQL, GQL, PLQ, LQG, PQV, VGQ, LQP, LQV, AQG, QPL, PQV, VGQ, and GQG; or
a tetrapeptide selected from LQGV (SEQ ID NO:10) and AQGV (SEQ ID NO:11).

50. The peptide of claim 49, wherein the autophagy inhibiting amino acid is selected from the group consisting of A, Q, L, and P.

51. A pharmaceutical formulation comprising the peptide of claim 45 and at least one pharmaceutically acceptable excipient.

52. A method of using the pharmaceutical formulation of claim 51 to treat a subject by lowering autophagy, modulating inflammation, modifying vascular permeability, improving tissue repair, and/or modulating an immune response.

53. A method for producing the peptide of claim 45, the method comprising: synthesizing the peptide with an automated peptide synthesizer.

54. A molecule comprising:
a chemotactic motif W selected from the group consisting of fMLP, WKYMVm (SEQ ID NO:6), xPGP (SEQ ID NO:16), AcPGP, SGP, AcSGP, YSFKDMQLGR (SEQ ID NO:3), and AcYSFKPMPLaR (SEQ ID NO:2); and
a peptide comprising amino acids selected from the group consisting of alanine (in one letter code: A), glutamine (Q), glycine (G), valine (V), leucine (L), isoleucine (I), proline (P) and arginine (R),
wherein the chemotactic motif W is connected to the peptide by a peptide bond.

55. The molecule of claim 54, wherein the peptide comprises:
a dipeptide selected from the group consisting of AQ, LQ, PQ, VQ and GQ;
a tripeptide selected from the group consisting of AQL, LQL, PQL, VQL, GQL, PLQ, LQG, PQV, VGQ, LQP, LQV, AQG, QPL, PQV, VGQ and GQG; and/or
a tetrapeptide selected from LQGV (SEQ ID NO:10) and AQGV (SEQ ID NO:11).


SEQUENCE LISTING (82 sequences)

SEQ ID NO: 1 - protein, 10 aa, Homo sapiens
ISHKDMQLGR

SEQ ID NO: 2 - protein, 10 aa, Artificial Sequence
  constrained human C5a analog
  MOD_RES (1)..(1): ACETYLATION
  VARIANT (9)..(9): is D-ala
YSFKPMPLXR

SEQ ID NO: 3 - protein, 10 aa, Artificial Sequence
  unconstrained sister analog C5a65-74465, F67++
YSFKDMQLGR

SEQ ID NO: 4 - protein, 4 aa, Artificial Sequence
  CXCR4 partial agonist
RSVM

SEQ ID NO: 5 - protein, 4 aa, Artificial Sequence
  CXCR4 superagonist
ASLW

SEQ ID NO: 6 - protein, 6 aa, Artificial Sequence
  FPR agonist
  VARIANT (6)..(6): is D-Met
WKYMVX

SEQ ID NO: 7 - protein, 5 aa, Artificial Sequence
  PPGPH
PPGPH

SEQ ID NO: 8 - protein, 5 aa, Artificial Sequence
  part of IL-8 sequence
ESGPH

SEQ ID NO: 9 - protein, 6 aa, Artificial Sequence
  analog of WKYMVm
WRWWWW

SEQ ID NO: 10 - protein, 4 aa, Artificial Sequence
  tetrapeptide
LQGV

SEQ ID NO: 11 - protein, 4 aa, Artificial Sequence
  tetrapeptide
AQGV

SEQ ID NO: 12 - protein, 21 aa, Artificial Sequence
  autophagy-inhibiting peptide
LPDISLKDLQFLQSFCPSEVQ

SEQ ID NO: 13 - protein, 6 aa, Artificial Sequence
  PG-domain amino acid motif
  VARIANT (1)..(1): Xaa can be any amino acid
  VARIANT (3)..(4): Xaa can be any amino acid
XGXXPG

SEQ ID NO: 14 - protein, 10 aa, Artificial Sequence
  chemoattractant motif
  MOD_RES (1)..(1): ACETYLATION
  VARIANT (9)..(9): is D-ala
YSFKPMPLXR

SEQ ID NO: 15 - protein, 4 aa, Artificial Sequence
  molecule specifically recognizing an CXC receptor
  VARIANT (1)..(1): X is an autophagy inhibiting amino acid
  VARIANT (4)..(4): X = 1-23 autophagy inhibiting amino acid(s)
XPGX

SEQ ID NO: 16 - protein, 4 aa, Artificial Sequence
  molecule specifically recognizing an CXC receptor
  VARIANT (1)..(1): X = 1-24 autophagy inhibiting amino acid(s)
XPGP

SEQ ID NO: 17 - protein, 4 aa, Artificial Sequence
  molecule specifically recognizing an CXC receptor
  VARIANT (4)..(4): X = 1-24 autophagy inhibiting amino acid(s)
PGPX

SEQ ID NO: 18 - protein, 5 aa, Artificial Sequence
  molecule specifically recognizing an CXC receptor
  VARIANT (1)..(1): X is an autophagy inhibiting amino acid
  VARIANT (5)..(5): X = 1-23 autophagy inhibiting amino acid(s)
XPGPX

SEQ ID NO: 19 - protein, 5 aa, Artificial Sequence
  chemoattractant motif comprising autophagy-inhibiting peptide
  VARIANT (1)..(1): X is no amino acid or Xaa can be any amino acid
  VARIANT (5)..(5): X is no amino acid or Xaa can be any amino acid
XPGPX

SEQ ID NO: 20 - protein, 5 aa, Artificial Sequence
  chemoattractant motif comprising autophagy-inhibiting peptide
  VARIANT (1)..(1): Xaa can be any amino acid
  VARIANT (5)..(5): Xaa can be any amino acid
XPGPX

SEQ ID NO: 21 - protein, 8 aa, Artificial Sequence
  synthetic peptide
AQGVAPGQ

SEQ ID NO: 22 - protein, 8 aa, Artificial Sequence
  synthetic peptide
LQGVAPGQ

SEQ ID NO: 23 - protein, 8 aa, Artificial Sequence
  synthetic peptide
AQGVLPGQ

SEQ ID NO: 24 - protein, 8 aa, Artificial Sequence
  synthetic peptide
LQGVLPGQ

SEQ ID NO: 25 - protein, 8 aa, Artificial Sequence
  synthetic peptide
AQGQAPGQ

SEQ ID NO: 26 - protein, 8 aa, Artificial Sequence
  synthetic peptide
LQGQAPGQ

SEQ ID NO: 27 - protein, 8 aa, Artificial Sequence
  synthetic peptide
AQGQLPGQ

SEQ ID NO: 28 - protein, 8 aa, Artificial Sequence
  synthetic peptide
LQGQLPGQ

SEQ ID NO: 29 - protein, 4 aa, Artificial Sequence
  Bacteria-derived N-formyl peptide
  MOD_RES (1)..(1): FORMYLATION
MIFL

SEQ ID NO: 30 - protein, 5 aa, Artificial Sequence
  Bacteria-derived N-formyl peptide
  MOD_RES (1)..(1): FORMYLATION
MIVIL

SEQ ID NO: 31 - protein, 5 aa, Artificial Sequence
  Bacteria-derived N-formyl peptide
  MOD_RES (1)..(1): FORMYLATION
MIGWI

SEQ ID NO: 32 - protein, 6 aa, Artificial Sequence
  Bacteria-derived N-formyl peptide
  MOD_RES (1)..(1): FORMYLATION
MIVTLF

SEQ ID NO: 33 - protein, 6 aa, Artificial Sequence
  Bacteria-derived N-formyl peptide
  MOD_RES (1)..(1): FORMYLATION
MIGWII

SEQ ID NO: 34 - protein, 9 aa, Artificial Sequence
  Bacteria-derived N-formyl peptide
  MOD_RES (1)..(1): FORMYLATION
MFEDAVAWF

SEQ ID NO: 35 - protein, 6 aa, Artificial Sequence
  Mitochondria-derived N-formyl peptide
  MOD_RES (1)..(1): FORMYLATION
MMYALF

SEQ ID NO: 36 - protein, 6 aa, Artificial Sequence
  Mitochondria-derived N-formyl peptide
  MOD_RES (1)..(1): FORMYLATION
MLKLIV

SEQ ID NO: 37 - protein, 9 aa, Artificial Sequence
  Mitochondria-derived N-formyl peptide
  MOD_RES (1)..(1): FORMYLATION
MYFINILTL

SEQ ID NO: 38 - protein, 6 aa, Artificial Sequence
  Mitochondria-derived N-formyl peptide
  MOD_RES (1)..(1): FORMYLATION
MFADRW

SEQ ID NO: 39 - protein, 6 aa, Artificial Sequence
  Mitochondria-derived N-formyl peptide
  MOD_RES (1)..(1): Nle
  MOD_RES (1)..(1): FORMYLATION
  MOD_RES (4)..(4): Nle
XLFXYK

SEQ ID NO: 40 - protein, 6 aa, Artificial Sequence
  Host-derived peptide D2D3
  VARIANT (6)..(6): is D-Pro
SRSRYX

SEQ ID NO: 41 - protein, 6 aa, Artificial Sequence
  Agonist from peptide library
WKYMVM

SEQ ID NO: 42 - protein, 5 aa, Artificial Sequence
  Agonist from peptide library
MMWLL

SEQ ID NO: 43 - protein, 5 aa, Artificial Sequence
  Agonist from peptide library
  MOD_RES (1)..(1): FORMYLATION
MMWLL

SEQ ID NO: 44 - protein, 6 aa, Artificial Sequence
  formyl-peptide receptor binding motif
  MOD_RES (1)..(1): FORMYLATION
MIVTFL

SEQ ID NO: 45 - protein, 11 aa, Artificial Sequence
  formyl-peptide receptor binding motif
  MOD_RES (1)..(1): FORMYLATION
MYVKWPWYVWL

SEQ ID NO: 46 - protein, 13 aa, Artificial Sequence
  formyl-peptide receptor binding motif
LESIFRSLLFRVM

SEQ ID NO: 47 - protein, 8 aa, Artificial Sequence
  formyl-peptide receptor binding motif
KWPWYVWL

SEQ ID NO: 48 - protein, 8 aa, Artificial Sequence
  formyl-peptide receptor binding motif
KWPWYIWL

SEQ ID NO: 49 - protein, 8 aa, Artificial Sequence
  formyl-peptide receptor binding motif
KWPWWVWL

SEQ ID NO: 50 - protein, 8 aa, Artificial Sequence
  formyl-peptide receptor binding motif
KWPWWIWL

SEQ ID NO: 51 - protein, 4 aa, Artificial Sequence
  autophagy-inhibiting-peptide
QQGV

SEQ ID NO: 52 - protein, 4 aa, Artificial Sequence
  autophagy-inhibiting-peptide
LQGV

SEQ ID NO: 53 - protein, 4 aa, Artificial Sequence
  autophagy-inhibiting-peptide
GQGV

SEQ ID NO: 54 - protein, 4 aa, Artificial Sequence
  autophagy-inhibiting-peptide
PQGV

SEQ ID NO: 55 - protein, 4 aa, Artificial Sequence
  autophagy-inhibiting-peptide
VQGV

SEQ ID NO: 56 - protein, 4 aa, Artificial Sequence
  autophagy-inhibiting-peptide
ALGV

SEQ ID NO: 57 - protein, 4 aa, Artificial Sequence
  autophagy-inhibiting-peptide
LLGV

SEQ ID NO: 58 - protein, 4 aa, Artificial Sequence
  autophagy-inhibiting-peptide
QLGV

SEQ ID NO: 59 - protein, 4 aa, Artificial Sequence
  autophagy-inhibiting-peptide
GLGV

SEQ ID NO: 60 - protein, 4 aa, Artificial Sequence
  autophagy-inhibiting-peptide
PLGV

SEQ ID NO: 61 - protein, 4 aa, Artificial Sequence
  autophagy-inhibiting-peptide
VLGV

SEQ ID NO: 62 - protein, 4 aa, Artificial Sequence
  autophagy-inhibiting-peptide
QAGV

SEQ ID NO: 63 - protein, 4 aa, Artificial Sequence
  autophagy-inhibiting-peptide
QLGV

SEQ ID NO: 64 - protein, 4 aa, Artificial Sequence
  autophagy-inhibiting-peptide
QGGV

SEQ ID NO: 65 - protein, 4 aa, Artificial Sequence
  autophagy-inhibiting-peptide
QPGV

SEQ ID NO: 66 - protein, 4 aa, Artificial Sequence
  autophagy-inhibiting-peptide
QVGV

SEQ ID NO: 67 - protein, 4 aa, Artificial Sequence
  autophagy-inhibiting-peptide
LAGV

SEQ ID NO: 68 - protein, 4 aa, Artificial Sequence
  autophagy-inhibiting-peptide
LGGV

SEQ ID NO: 69 - protein, 4 aa, Artificial Sequence
  autophagy-inhibiting-peptide
LPGV

SEQ ID NO: 70 - protein, 4 aa, Artificial Sequence
  autophagy-inhibiting-peptide
LVGV

SEQ ID NO: 71 - protein, 12 aa, Artificial Sequence
  synthetic peptide
KWPWYIWLAQGV

SEQ ID NO: 72 - protein, 14 aa, Artificial Sequence
  synthetic peptide
KWPWYIWLAQLPGP

SEQ ID NO: 73 - protein, 16 aa, Artificial Sequence
  synthetic peptide
KWPWYIWLQGVLPALP

SEQ ID NO: 74 - protein, 15 aa, Artificial Sequence
  synthetic peptide
KWPWYIWLQGLQPGQ

SEQ ID NO: 75 - protein, 14 aa, Artificial Sequence
  synthetic peptide
WPWYIWLQGLQPGQ

SEQ ID NO: 76 - protein, 11 aa, Artificial Sequence
  synthetic peptide
  MOD_RES (1)..(1): FORMYLATION
MLFLQGLQPGQ

SEQ ID NO: 77 - protein, 11 aa, Artificial Sequence
  synthetic peptide
  MOD_RES (1)..(1): ACETYLATION
PGPAQGLQPGQ

SEQ ID NO: 78 - protein, 11 aa, Artificial Sequence
  synthetic peptide
  MOD_RES (1)..(1): ACETYLATION
PGPLQGLQPGQ

SEQ ID NO: 79 - protein, 11 aa, Artificial Sequence
  synthetic peptide
  MOD_RES (1)..(1): ACETYLATION
PGPAQGVAPGP

SEQ ID NO: 80 - protein, 11 aa, Artificial Sequence
  synthetic peptide
  MOD_RES (1)..(1): FORMYLATION
MLFLQGVAPGP

SEQ ID NO: 81 - protein, 17 aa, Artificial Sequence
  synthetic peptide
  MOD_RES (1)..(1): ACETYLATION
  VARIANT (9)..(9): is D-Ala
YSFKPMPLXRAQGVLPG

SEQ ID NO: 82 - protein, 24 aa, Artificial Sequence
  synthetic peptide
  MOD_RES (1)..(1): ACETYLATION
  VARIANT (9)..(9): is D-Ala
YSFKPMPLXRAQGVLPGLQGVLPG